SEQUENCE LISTING (15 sequences)

SEQ ID NO: 1 - DNA, 35712 bp, Artificial Sequence
  recombinant adenovirus (delta923-931ADV5/ASCHK1)
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcaatcgatttccaagggttgaggtatgtttttttttctttccagtcagaatactcctgacagctgtcactgggttggtcccatggcaattctccagcgagcattgcagtaagtactattccacaggaccaaacatcaactggttctgcatgaaattctcttctcttcagaagttctggagcaacatatggtaaagtaccacacatcttgttcaacaaacgctcacgattattataccgaaatactgttgccaagccaaagtctgagattttgaggttatccctttcatccaacagaagattttctggtttaatatccctgtgagttattccaataccatgcagataaaccacccctgccatgagttgatggaagaatctctgagcatctggttcaggcatgcctatgtctggctctattctgtcaaaaagctctcctccactacagtactccagaaataaatattggatattgccttctctcctgtgaccatagaattttactacattttcatgatttagcattttattgatacagatctctttcttaatattttctggacagtctacggcacgcttcatatctacaatcttcactgcgactgcttcttcaatcgatactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 2 - DNA, 35909 bp, Artificial Sequence
  recombinant adenovirus (delta923-931ADV5/ASPLK1)
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcaatcgatgcagctcgttaatggttgggcgggcgatgggatctgtctgaagcatctcttggatgagggaggcggccacggggttgatgtgcttgggaatactgtattcattcttcttgatccggaggtaggtctcttttaggcaagaagtctcaaaaggtggtttgcccactaacaaggtatacatgatacacccaatggaccacacatccacctcgaaactgtgccctttcttgctcagcacctcgggagctatgtaattaggagtcccacacagggtcttcttcctctccccgtcatattcgactttggttgccagtccaaaatcccctattttcacctccagatcttcattcaggaaaaggttgcccagcttgaggtctcgatgaataactcggtttcggtgcaggtactggcagccaagcacaatttgccgtaggtagtatcgggcctcaggctcagtcagggctttcctcctcttgtgcagctccaggagagacctccggcggcagagctccaacaccacgaacacgaagtcgttgtcctcgaaaaagccgtggaatcctacgacgtgctggtgggcgaggctgcggtgaatggatatttccatggacatcttctccctctggtgcggcttgagcagcagagacttaggcacaatcttgcccgcgaacacctccttggtgtccgcgtccgagatctcgaagcacttggcaaagccgcccttgcccaaaaagcggccccgcacatagcgccgccggctgcgtgggtccactaggacctccgggatctctttcgccggtggagccatcgatactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 3 - DNA, 22 bp, Artificial Sequence
  Primer 1
cgggatccgggcccccatttcc

SEQ ID NO: 4 - DNA, 30 bp, Artificial Sequence
  Primer 2
gtcactgggtgggatcacctccggtacaag

SEQ ID NO: 5 - DNA, 30 bp, Artificial Sequence
  Primer 3
ccggaggtgatcccacccagtgacgacgag

SEQ ID NO: 6 - DNA, 23 bp, Artificial Sequence
  Primer 4 uses in the construction of PXC1 Mutant
tgctctagacacaggtgatgtcg

SEQ ID NO: 7 - DNA, 20 bp, Artificial Sequence
  Primer sequence for assaying
agccggagcagagagccttg

SEQ ID NO: 8 - DNA, 20 bp, Artificial Sequence
  Primer 1 used in the deletion of a 28532-29360 region from ADV5
gggtcaacggaatccgcgcc

SEQ ID NO: 9 - DNA, 36 bp, Artificial Sequence
  Primer 2 used in the deletion of a 28532-29360 region from ADV5
cccacatagagtatcgattgcgcctttggcctaata

SEQ ID NO: 10 - DNA, 41 bp, Artificial Sequence
  Primer 3 used in the deletion of a 28532-29360 region from ADV5
gccaaaggcgcaatcgatactctatgtgggatatgctccag

SEQ ID NO: 11 - DNA, 21 bp, Artificial Sequence
  Primer 4 used in the deletion of a 28532-29360 region from ADV5
ggggaacaaaacgcagatagg

SEQ ID NO: 12 - DNA, 28 bp, Artificial Sequence
  Upstream primer used for PCR
ccatcgatctgaagaagcagtcgcagtg

SEQ ID NO: 13 - DNA, 28 bp, Artificial Sequence
  Downstream primer used for PCR
ccatcgatttccaagggttgaggtatgt

SEQ ID NO: 14 - DNA, 28 bp, Artificial Sequence
  Upstream primer used for PCR
ccatcgatggctccaccggcgaaagaga

SEQ ID NO: 15 - DNA, 28 bp, Artificial Sequence
  Downstream primer used for PCR
ccatcgatgcagctcgttaatggttggg

(19) United States
(12) Patent Application Publication
(10) Pub. No.: US 2005/0079158 A1
(43) Pub. Date: Apr. 14, 2005

(54) Construct of anti-cancer recombinant adenovirus, method for preparing the same and use thereof

(75) Inventors: Jianfeng Zhou, Guangdong (CN); Ding Ma, Guangdong (CN); Yunping Lu, Guangdong (CN); Shixuan Wang, Guangdong (CN); Gang Chen, Guangdong (CN); Qinglei Gao, Guangdong (CN)

Correspondence Address: SEED INTELLECTUAL PROPERTY LAW GROUP PLLC, 701 FIFTH AVE, SUITE 6300, SEATTLE, WA, 98104-7092, US

(73) Assignee: Shenzhen Allucks BioTech Co., Ltd., Guangdong (CN)

(21) Appl. No.: 10/860,630

(22) Filed: Jun. 3, 2004

(30) Foreign Application Priority Data
Jun. 5, 2003 (CN) CN03126758.0
Jun. 5, 2003 (CN) CN03126759.9

(51) Int. Cl. A61K 48/00; C12N 7/00; C12N 15/861

(52) U.S. Cl. 424/93.2; 435/456; 435/235.1

(57) ABSTRACT

Disclosed is a recombinant human adenovirus type 5 adenovirus construct, in which a fragment in the E1A region of the ADV5 genome and a fragment in the E3 region are deleted while a foreign cDNA fragment is reversely inserted into the deleted E3 region. A method for preparing the recombinant ADV5 construct is also provided. The construct provided herein presents a tumor-specific replication, tumor-specific expression of the inserted anti-gene and tumor-specific bystander effects, and is suitable for use in tumor therapy.

### CROSS REFERENCE OF RELATED APPLICATIONS

The present application claims the benefit of Chinese patent application 03126758.0 filed on Jun. 5, 2003 and entitled “Preparation of an Anti-cancer Recombinant Adenovirus Construct Inactivating Selectively the Tumour Cell PLK1 and Use Thereof”, and Chinese patent application 03126759.9 filed on Jun. 5, 2003 and entitled “Preparation of an Anti-cancer Recombinant Adenovirus Construct Inactivating Tumour CHK1 Genes and Use thereof”, which are explicitly incorporated herein by reference in their entirety.

### BACKGROUND OF THE INVENTION

1. Field of the Invention

The present invention relates to a recombinant adenoviral construct, in particular, to a human adenovirus type 5 (ADV5) recombinant construct, in which sequences in the E1A region and in the E3 region are deleted and a foreign cDNA fragment is inserted into the deleted ADV5 E3 region in a reverse orientation. Furthermore, the present invention also provides a method for the preparation of the recombinant-construct and uses of the recombination construct.

2. Background of the Invention

Malignant tumors are one of diseases significantly harming human beings. According to the latest epidemiological investigation, there have been more than 3 million cancer patients in China. More than one million patients with cancer die every year. Meanwhile, 1.6 to 2.0 million of new cases of malignant tumor are diagnosed each year and the number of patients at new diagnosis increases with a rate of 3% per year.

A number of therapeutical agents have been developed to treat various caners. However, most of therapeutical agents used face an austere challenge due to their failure to eradicate malignant tumor. Therefore, it is urgent to develop novel anticancer approaches against cancers to overcome drawbacks in the prior art.

Modern biotechnology and deeper understanding of molecular mechanisms underlying tumor genesis have brought about many breakthroughs of molecular medicines in both theory and methodology. As a result, many new strategies against cancer are coming into shape. For example, a new medicine for treatment of chronic myelogenous leukemia, Gleevec, has been approved as a clinical prescription drug. Moreover, clinical trials of a great number of molecular target-specific anti-cancer compounds indicate that a new era of cancer therapy is coming. Molecular target therapy is to identify a molecule which is crucial for the survival of cancer cells and then, target the molecule to kill cancer cells. This would also be a basal strategies against tumors in the future. Up to the end of 2001, FDA had approved more than 3,000 molecular therapeutical agents into clinical trials, including cell signal transduction pathway inhibitors, anti-angiogenesis agents, recombinant monoclonal antibodies and gene therapy, which have brought about new hopes for cancer patients. Control of human tumors in the future essentially depends on the development and improvement of the molecular therapy.

Modern molecule-target therapy falls into three main categories: monoclonal antibodies, small molecular compounds and gene therapy.

Gene therapy, one of the most vigorous fields in the modern medicine, has been widely used in clinical treatment for various tumors and shown a prosperous future. From 1989 to 2000, the Center for Biologics Evaluations and Research (CBER) authorized more than 350 experimental projects for clinical gene therapy. 70% of the protocols are for tumor gene therapy, some of which had entered phase III clinical trial and shown promising. With the ever-increasing medical demands, gene therapy agents will be expected to enter the medicine market in a few years as drugs for clinical therapy, and become an indispensable part of the tumor therapy system.

Adenoviral vectors have long been used in cancer gene therapy and become one of the most widely used vectors in tumor gene therapy due to its clinical feasibility and safety. The adenoviral vectors have the following advantages:
(1) Broad spectrum of infection: The adenoviral vectors can transduce either quiescent or proliferating tumor cells of different tissue origins quiescence or proliferation;
(2) Convenience in clinical applications: They can be administered via lacuna (abdomen, thorax and head), direct tumor injections or systemic delivery, etc;
(3) Better safety: ADV-TK life cycle does not normally involve integration into the host genome, rather they replicate as episomal elements in the nucleus of the host cell and consequently there is no risk of insertion mutagenesis;
(4) Short duration: Transgene expression of ADV-TK in cells was typically transient, lasting only a few weeks or months, which is especially suitable for tumor treatment; and
(5) Easy preparation: The adenovirus vectors in the clinic scale can be readily prepared and purified at high concentrations.

At present, most of adenovirus vectors used in clinical trial are replication-defective because E1 and E3 regions are deleted. The killing effects on the tumor always rely on the concentration of adenovirus administrated and efficiency of transfection. Therefore, the killing effects of the adenovirus on tumor cells far from the injection site is always insufficient, tumor replication-permissive adenovirus vectors, on the other hand, are able to overcome the drawbacks characterized of first generation adenovirus vectors. The so-called tumor replication-permissive adenovirus vectors imply vectors that can selectively replicate in tumor cells but not in normal cells. That is, the adenovirus can be produced in the tumor cells, and thereby to lyse tumor cells. When the adenovirus is released from a lysed tumor cell, it can further infect more other tumor cells relatively far from the injection site. The cycles will last for other several cycles. As a result, tumor cells far from the injection site can also be effectively killed, and the treatment gains better killing efficiency. Moreover, this vector will have minor effects on normal cells because it cannot replicate in normal cells. Papers in certain well-known medical journal comment these adenovirus mutants as “smart bomb” against tumor cells. In the 21st MCR Annual Meeting, a research report from the Anderson Tumor Center Houston indicates that the replication deficient adenovirus distributes in human malignant tumor tissues only in a limited areas around the injected sites, and thus affecting the killing effects of the adenovirus mutants. The adenovirus ONYX-015 that can specifically replicate in tumor cells can diffuse throughout the whole tumor sites by replication, and thus generating prominent killing effects.

ONYX-015 is an adenovirus developed by ONYX Pharmaceuticals Inc. that can selectively replicate in tumor cells. ONYX-015 is constructed by partially deleting an E1 B 55 kDa coding sequence in wild-type human adenovirus 5 genome, adding thereto a translation termination signal, but reserving a coding sequence of 19 kDa E1B. Under normal conditions, the replication and proliferation of the wild-type adenovirus relies on the inhibiting effects of E1B 55 kDa protein on the function of p53 protein. ONYX-015 lacking E1B 55 kDa protein cannot replicate in normal cells carrying wild-type p53 gene. To the contrary, since the mutation of p53 gene in malignant tumor patients is as frequent as 50-70%, ONYX-015 can replicate in tumor cells and lyse the cells at last. Therefore, ONYX-015 possesses a high selectivity on killing tumor cells but does no harm to normal cells.

There have been 4 patent applications in China related to ONYX-015.

Chinese patent application No. 98113494.7 entitled “Apoptosis-inhibiting gene virus construct and use in tumor gene therapy” discloses a virus construct prepared by partially deleting an E1B 55 kDa coding sequence and inserting a marker gene LacZ in the deleted region. The construct also has a deletion in the E3 region.

Chinese patent application No. 99124030.8 entitled “Deficient virus and method for constructing the same” discloses a construct obtained by deleting a coding sequence of E1B 55 kDa in 2809-3329 bp region and adding a termination codon to the deletion region.

Chinese patent application No. 01144628.5 entitled “Preparation of recombinant adenovirus construct deleting E1A coding sequence and use thereof” discloses a recombinant adenovirus construct that deletes an E1A coding sequence (382-1630 nt) and fails to express E1A functional proteins.

Chinese patent application No. 01144629.3 entitled “Recombinant adenovirus construct deleting of both 19 kDa and 55 kDa E1B coding sequences and use thereof” discloses a recombinant adenovirus construct incapable of expressing E1B functional proteins prepared by deletion of both 19 kDa and 55 kDa E1B coding sequences.

Compared with the replication-defective adenovirus vectors (so called the first generation of ADV5 gene therapy vectors), the present “tumor replication-permissive adenovirus vectors” (the second generation of ADV5 gene therapy vectors) have made prominent progress in both therapy theory and gain practical improved killing effects. However, the second generation of ADV5 gene therapy vectors still have some common shortcomings such as: lower replication efficiency as compared with the wild-type ADV5 for the reason that modification to adenovirus give rise to less replicated potential than wild-type ADV5, and thus impede the killing effects. As can be denoted from the facts that response to ONYX-015 administration is between 0-14%, when used solely. The choice of promoters used in transgene cassette in previous recombinant ADV5 mutants, usually adapted virus promoter such as CMV or SV40, is also problematic, because these kinds of virus promoters exhibit strong non-selective promoter activities both in malignant cells and normal cells, and hence generates unwanted transgene expression in normal cells and narrows down therapeutic windows.

To date, numerous molecular targets inside tumor cells have been identified. Based on these kinds of targets, some novel anticancer approaches have been developed and more others are already under testing in clinical trials. Despite this fundamental progression, one major challenge still facing the modern pharmaceutical companies and oncologists as well is that these targets, although better than previous ones, are still lacking tumor specificity, therefore, more ideal methodology that can target crucial molecules in tumor cells other than normal cells need be developed urgently.

### SUMMARY OF THE INVENTION

The present invention provides a recombinant human adenovirus type 5 (ADV5) construct that comprises two deletions: one in the E1A region and the other in the E3 region and a foreign cDNA fragment inversely inserted into the latter deletion. The deletion in the E1A region encodes an E1A protein that is incapable of binding to retinoblastoma (Rb) protein, but still capable of inactivating transcription. The deletion in the E3 region allows an insertion of a foreign gene in a reverse orientation to be under the control of the endogenous promoter in the E3 region, resulting in the production of an antisense transcript of the foreign gene. In certain embodiments, the deletion in the E1A region is from nucleotide No. 923 to nucleotide No. 931 of the ADV5 genome. In certain embodiments, the deletion in the E3 region is from nucleotide No. 28532 to nucleotide No. 29360 of the ADV5 genome. In certain embodiments, the foreign cDNA fragment is a gene necessary for cell survival, such as CHK1, PLK1, or fragments thereof (e.g., a fragment corresponding to nucleotide Nos. 853-250 of CHK1 mRNA and a fragment corresponding to nucleotide Nos. 960-161 nt of PLK1 mRNA).

The present invention also provides a method for preparing the recombination ADV5 construct, which comprises deleting a fragment in the E1A region of an ADV5 genome, deleting a fragment in the E3 region of the ADV5 genome, and inversely inserting a foreign cDNA fragment into the deleted E3 region.

In an embodiment of the invention, an enzyme cleavage site such as a ClaI is introduced to the deleted ADV5 E3 region.

In addition, the present invention provides a method of treating a tumor in a subject comprising administrating to a subject having a tumor a therapeutically effective amount of a recombinant ADV5 construct disclosed herein. Further, the present invention provides a method for inhibiting tumor metastasis in a subject comprising administering to a subject having a tumor a therapeutically effective amount of a recombinant ADV5 construct disclosed herein.

The recombinant construct of the present invention shows good tumor-selective replication, tumor-specific expression of inserted anti-sense gene and tumor-specific by-stander effects. It also has a high therapeutic effect on many tumors via intratumal injection or intravenous administration, without affecting gene expression of normal cells. The construct of the present invention is suitable for use in tumor therapy.

### BRIEF DESCRIPTION OF THE DRAWINGS

FIG. 1 is a sketch map of a plasmid pXC1 that comprises a 21-5790 nt sequence of ADV5;

FIG. 2 shows a sketch map of a plasmid pBHGE3 that comprises all the genomic sequence of ADV5 except that a ADV5 packaging signal sequence (194-358 nt) where an artificial sequence is inserted, with a length of 37436 bp;

FIG. 3 is a diagrammatical view of the ADV5 E3 region that encodes 7 types of proteins, deleting a 28530-29355 bp sequence of E3 6.7 k/gp 19 k region, in which a foreign cDNA fragment is inversely inserted;

FIG. 4 shows a sketch map of pCDNA3.1 plasmid used for constructing an intermediate vector pCDNA3.1-ΔE3;

FIG. 5 shows a sequence listing of a recombinant adenovirus Δ923-931ADV5/ASCHK of the present invention; and

FIG. 6 shows a sequence listing of a recombinant adenovirus Δ923-931ADV5/ASPLK1 of the present invention.

### DETAILED DESCRIPTION OF THE INVENTION

It is understood that in one hour after entering cells, ADV5 will express the E1A functional protein. This performance has significance in biology. Firstly, E1A protein binds to retinoblastoma mutant gene (Rb) protein to make an endogenous transcription factor E2F released from a complex Rb-E2F in a host cell. And at the same time, E1A protein will mobilize ADV5 to express E1B functional protein so as to inactivate an endogenous transcription factor p53's inhibition upon Cyclin-Dependent Kinase (CDK). With the impetus of E1A, E1B and E2F, the host cell enters into a DNA synthesis period, which serves as a prerequisite for self-replication of adenovirus genome. Secondly, E1A protein activates the expression of ADV5 early genes EIB, E2, E3 and E4, and late genes L1-6, which provides a possible environment for the whole life cycle of ADV5 including self-replication of adenovirus genome, packaging of adenovirus particles, escaping cellular immune elimination of the host cell, lysing the host cell, releasing the adenovirus particles and re-infecting another host cell. It can be seen that ADV5, dependent on E1A protein, initiates the sequential life cycle, thereby lysing the cells at last.

Located in 560-1112 nt and 1229-1545 nt of ADV5 genome, the coding sequence of E1A functional protein includes three sequences: a 677-799 nt, E1A conservative sequence 1 (CR1) for binding a transcription cofactor P₃₀₀; a 917-979 nt, E1A conservative sequence 2 (CR2) for binding a retinoblastoma mutant gene (Rb); and a 1007-1115 nt, conservative sequence 3 (CR3) serving as a gene transcription activation region.

It was hypothesized that almost all tumors have defects related to the Rb regulation pathway. If the CR2 region of E1A 917-979 nt is modified by deletion to inactivate Rb binding characteristic of E1A protein while keeping transcription activation of E1A, it is possible that an adenovirus recombinant construct acquired can thereby be effectively replicated in tumor cells, and ultimately kills the tumor cells. On the other hand, the normal cells possess a normal Rb regulation pathway, which can effectively prevent the adenovirus recombinant construct from initiating the sequential life cycle with E1A protein, leading only to a septic abortion (see, Fueyo et al., Oncogene, 2000, 19:2-12; Heise et al., Nature Medicine, 2000, 10:1134-1139).

As indicated above, the present invention provides a recombinant ADV5 construct with a deletion in the E1A region. Such a deletion will result in the production of a mutant E1A that retains the transcription activation activity, but not the capability of binding to Rb, of a wild type E1A protein. The deletion is usually within the region from nucleotide No. 917 to nucleotide No. 979 (used herein exchangeably with “a 917-979 nt sequence”) of the ADV5 genome. For instance, the deletion may be from nucleotide No. 923 to nucleotide No. 931 of the ADV5 genome (i.e., CTT ACC TGC, coding amino acid Nos. 122-124 of E1A protein).

The recombinant ADV5 of the present invention also contains a deletion in the E3 region. Such a deletion allows for an insertion of a foreign DNA in a reverse orientation, which expresses an anti-sense mRNA under the control of the endogenous promoter in the E3 region of ADV5. The endogenous promoter has a similar high activity to the CMV promoter. The switch-on of the promoter depends on the adenovirus replication in cells, and if the adenovirus does not replicate, the foreign anti-sense mRNA will not be expressed. In certain embodiments, the deletion in the E3 region is at nucleotide Nos. 28530-29355 of the ADV5 genome.

The foreign gene used in the invention is a cDNA fragment that is inversely inserted into the deleted ADV5 E3 region.

The foreign cDNA fragment is selected from those necessary for cell survival, such as PLK1, CHK1, STAT3 and STAT5. Examples of foreign cDNA fragments used in the present invention include, but not limited to, a CHK1 cDNA fragment and a PLK1 cDNA fragment. Preferably, the CHK1 cDNA fragment is a 620 bp CHK1 cDNA fragment (equivalent to 853-250 nt of CHK1 mRNA), and the PLK1 cDNA fragment is a 820 bp foreign cDNA fragment of PLK1 (equivalent to 960-161 nt of PLK1 mRNA).

In certain embodiment, an enzyme cleavage site is introduced into the 28532-29360 nt deletion region of ADV5 E3 region of the construct in the invention. The foreign cDNA fragment can be easily introduced to the enzyme cleavage site. Preferably, the enzyme site is ClaI.

CHK1 gene is the abbreviation of “Cell Cycle Checkpoint Kinase 1”, which is a key activation gene in DNA damage repair/cell cycle checkpoint. PLK1 gene is the abbreviation of polo-like kinase 1 which is a key regulatory kinase in cell mitotic checkpoint. The function of polo-like kinase 1 is to facilitate entry of cells and to exit mitosis. PLK1 expresses abnormally in most of tumor cells and relates to genomic instability of tumor cells. It has been proved that CHK1 and PLK1 are crucial genes for cell survival. Inactivating CHK1 or PLK1 will lead to apoptosis. In the present invention, the two genes are preferable molecular drug targets.

Another aspect of the present invention is to provide a method for preparing an adenovirus recombinant construct, which comprises the steps of deleting a 923-931 nt sequence of ADV5 genome; deleting a 28532-29360 nt from an ADV5 E3 region and inversely inserting a foreign cDNA fragment in the latter deleted region.

In the method of the invention, a ClaI enzyme cleavage site is preferably introduced into the deleted region where the foreign cDNA fragment will be inserted.

In an embodiment of the method for preparing the recombinant construct according to the present invention, the method includes the steps of:
a) deleting a 923-931 nt sequence from the E1A coding region of a plasmid pXC1 to form a first vector;
b) co-infecting a first cell with the first vector and pBHGE3;
c) extracting a DNA containing terminal proteins from the infected cell and digesting the DNA with EcoRI to obtain a first fragment;
d) deleting a 28532-29360 nt sequence from the E3 region of an ADV5 and inserting a enzyme cleavage site in the deleted region to form a second vector;
e) inversely inserting a foreign cDNA fragment in the enzyme cleavage site to form a third vector;
f) digesting the third vector with EcoRI to obtain a second fragment; and
g) co-infecting a second cell with the first fragment and the second fragment to obtain the recombination adenovirus construct that expresses functional proteins of an E1A mutant.

The deleting step used in the invention is performed according to Kunkel's method (Kunkel T A, Roberts J D, Zakour R A, et al, Rapid and efficient site-specific mutagenesis without phenotypic selection, Methods in Enzymology, 1987, 154: 167˜182).

Although not wishing to be bound to any specific hypotheses, the inventors believe that the selective killing of tumor cells by the recombination construct of the present invention are likely based on a triple mechanism including greatly increasing copies of foreign anti-sense cDNA due to the replication of the recombination construct only in tumor cells; avoiding the attack to normal cells while specifically inactivating gene expression of tumor cells; and forming the strong bystander effects because the recombination construct uses tumor cells as the host cell for replication, producing a higher concentration of the adenovirus in tumors.

### EXAMPLES

The present invention will be described in detail with reference to the following examples, which will not be considered to limit the scope of the present invention. Unless otherwise specially indicated, all enzymes and PCR primers used herein are from Gibco Inc., USA.

### Example 1

### Construct of pXC1 Mutant (Δ923-931pXC1)

1. Materials

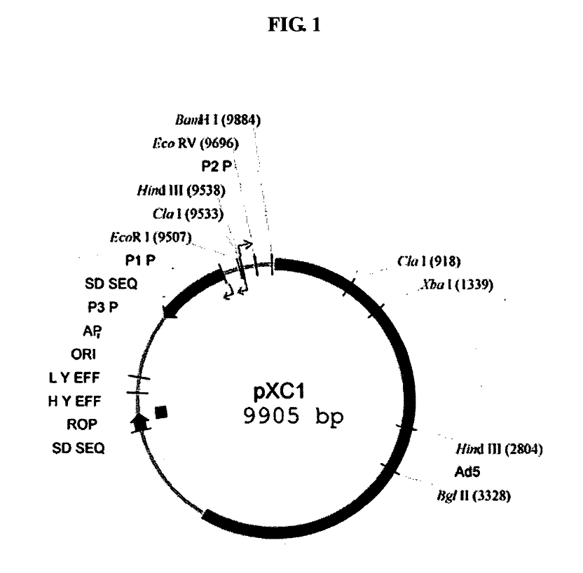

Plasmid pXC1: provided by Microbix Biosystem Inc. (Toronto, Ontario, Canada, Catalogue No. PD-01-03), comprising a 21-5790 nt sequence of human adenovirus type 5 (ADV5), whose sketch map is shown in FIG. 1, in which the 194-358 nt sequence is a packaging signal of ADV5, the 560-1112 nt and 1229-1545 nt sequences are coding sequences of E1A functional protein, the 9883-9888 nt sequence involves a BamHI cleavage site, and the 1338-1343 nt sequence involves an XbaI cleavage site.

Primer 1: 5′-CG GGA TCC GGG CCC CCA TTT CC-3′ (the underlined part being a BamH cleavage site).

Primer 2: 5′-GTCACTGGGTGGGATCAC CTC CGG TAC AAG-3′ (the underlined part being complementary to primer 3).

Primer 3: 5′-CCG GAG GTG ATC CCA CCC AGT GAC GAC GAG-3′ (the underlined part being complementary to primer 2).

Primer 4: 5′-TGC TCT AGA CAC AGG TGA TGT CG-3′ (the underlined part being an XbaI cleavage site).

2. Deleting 923-931 nt with 3-Time PCR

2.1. Preparation of Fragment 1

A 100 μl reaction mixture was prepared by mixing pXC1 as a DNA template, 10 μl of a 10×PCR buffer containing MgCl₂, 10 μl of 2 mM dNTP, 1 μl of 10 μM primer 1, 1 μl of 10 μM primer 2, 2.5 u of HiFi Taq polymerase and water, in which the concentration of pXC1 was 10 ng/μl.

The PCR conditions were as follows: initial denaturation at 95° C. for 30 seconds, 95° C. for 45 seconds, 60° C. for 1 minute, 72° C. for 2 minutes for a total of 28 cycles, and final extension at 72° C. for 10 minutes. A product with a 940 bp (Fragment 1) was obtained, after conventional gel electrophoresis purification.

2.2. Preparation of Fragment 2

A PCR was performed as the same as in the preparation of Fragment 1 except using primer 3 and primer 4 instead of primer 1 and primer 2. Fragment 2 was obtained with a 400 bp, after conventional gel electrophoresis purification.

2.3. Preparation of Fragment 3

A PCR was performed by mixing 2 μl of Fragment 1 (50 ng) with 1 μl of Fragment 2 (25 ng) as a template. Primer 1 was used as the upstream primer, and Primer 4 was used as the downstream primer. The PCR conditions were the same as described above. After purification with a QIAquick 8 PCR purification kit (QIAGEN, German, Cat. No. 28142), the PCR product was digested with BamHI and XbaI overnight and then separated by electrophoresis in 1% agarose gel. Fragment 3 was obtained with a 1,400 bp for cloning.

3. Preparation of pXC1 Mutant

pXC1 was digested with BamHI and XbaI overnight. By electrophoresis in 1% agarose gel, two fragments with 1,400 bp and 8,500 bp were obtained. A reaction of 40 ng of the 8500 bp fragment and 90 ng of Fragment 3 was performed with a DNA T₄ ligase. 1.5 μl of the resultant was transformed into 100 μl of DH5α competent cells. The positive colonies were selected and the plasmids were micro-extracted. DNA sequencing was used to identify the pXC1 plasmid mutant Δ923-931pXC1 that has been deleted the 121-129AA 923-931 nt sequence and the plasmid was used to clone recombinant adenovirus.

### Example 2

### Construction of Δ923-931ADV5 Recombinant Adenovirus

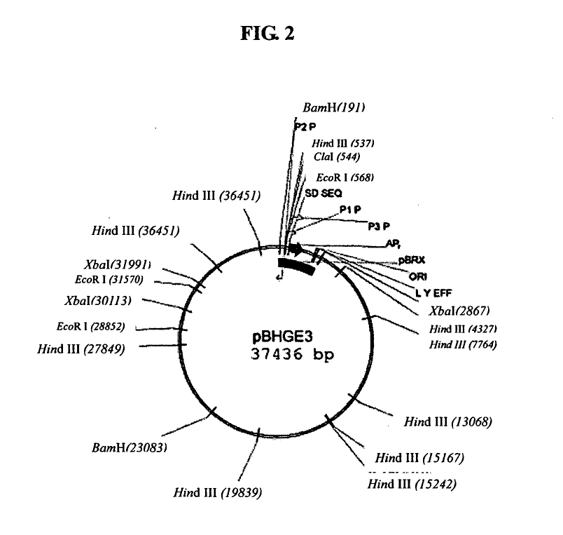

pBHGE3 plasmid was purchased from Microbix Biosystem Inc. (Toronto, Ontario, Canada, Catalogue No. PD-01-12). The plasmid comprises all the ADV5 genomic sequences except that an artificial sequence was used instead of the sequence of 194-358 nt (ADV5 packaging signal). The whole length of the plasmid was 37436 bp whose sketch was shown in FIG. 2.

Preparation of Δ923-931ADV5 Recombinant Adenovirus Constructs

(1). 7.5×10⁵ of 293 cells (ATCC, U.S.A., Catalogue No.: CRL-1573) were seeded in a 15 cm culture plate with a 10% FBS DMEM culture medium. The number of the cells reached 1-1.5×10⁶ next day, of which almost 70% was confluent.

(2). Preparation of Calcium Phosphate Solution for Co-Infection

1600 μl of a sterilized 2×HBS solution containing 42 μg of the pBHGE3 and 42 μg of the Δ923-931pXC1 prepared as in Example 1 was prepared, which includes 280 mM NaCl, 43 mM HEPES, 10 mM KCl, and 10 mM Na₂HPO₄.7H₂O, 2% dextrose, with pH7.05-7.15. To the resultant solution was added sterilized distilled water to make the final volume to reach 2840 μl. After fully mixed, 50 μl of 2.5M CaCl₂ was added slowly to the solution before letting DNA/CaCl₂ precipitate for 45-60 minutes at room temperature to obtain a solution for co-transfection.

(3). 500 μl of the solution for co-transfection was added to a 60 mm culture plate containing 5 ml of the 293 cells solution from the above 15 cm culture plate with a 10% FBS DMEM culture medium which was refreshed 3-4 hours before used. The cells were incubated in 5% CO₂ at 37° C. for 4-6 hours. The supernatant was discarded. The cells pellets were washed with PBS.

(4). The cells were treated with a DMEM containing 15% glycerol, and washed with PBS. A complete medium was used to replace the original medium.

(5). 10% Agarose (Low Melting Point) PBS was prepared and autoclaved, aliquoting into 10 ml tubes. The Agarose PBS was melted in boiled water and kept at 45° C. Before using, 30 ml 10% FBS DMEM was added to it with the final concentration of 1.2%.

(6). The complete medium was removed from the cell solution. 5 ml of a 1.2% agarose (low melting point) PBS prepared in (5) was added into the plate, and another 3 ml of 1.2% agarose (low melting point) PBS was added into the plate every 4-5 days.

(7). After 14-21 days, plaques were observed. 6-12 plaques were selected and transferred to a 24-well plate, each well of which contained 0.5 ml 10% FBS DMEM, and then filtered for 24 hours at 37° C.

(8). 1×10⁵ 293 cells were seeded in a 24-well culture plate with a 10% FBS DMEM medium. The cell number was 2×10⁵ next day. Almost 70% of the cells were confluent. The supernatant was discarded. 100 μl of the filtrate (containing about 10³ adenoviruses) prepared in (7) was added to each well and the plate was shaken gently 3 times, and the cell mixture was incubated in 5% CO₂ at 37° C. for 90 minutes.

(9). A complement culture medium was added to the incubated mixture to make the volume 1 ml. And the cells were incubated in 5% CO₂ at 37° C. for 5-10 days till the complete CPE appeared. The so-called CPE means cell poison effects, that shows the cell, mainly comprising nucleolus, turned round and were floating. If the complete CPE didn't appear after 10 days, it meant that potency of the adenovirus was so low that a second amplification was needed.

(10). The plate was frozen/thawed for three rounds to release the adenoviruses. Lysed solutions in the wells were collected in a 15 ml tube and centrifuged for 10 minutes. The supernatant was collected and frozen at −80° C. Resultant liquid contained about 5×10⁷ adenoviruses/ml (2nd generation adenovirus).

(11). Re-Amplification of the 2nd Generation Adenovirus

5×10⁶ 293 cells were seeded in a 75 cm² culture plate with 10 ml 10% FBS DMEM medium. 3-4 hours before transfection, the medium was refreshed.

1 μl of the liquid containing the second generation adenovirus was mixed with a complement culture medium until 1 ml (MOI being about 5). The mixture was then added to the 75 cm² culture plate from which the liquid was discarded previously. The plate was shaken 3 times and incubated in 5% CO₂ at 37° C. for 90 minutes following by adding 9 ml 10% FBS DMEM. The mixture was incubated in 5% CO₂ at 37° C. for 4-7 days.

(12). Since 293 cell genome contained complete E1A genes, it was easily contaminated when extracting the positive adenovirus DNA, thereby leading failure to identification. Therefore, the Δ923-931ADV5 was re-amplified in Hela tumor cells (ATCC, USA, Catalogue No.: CCL-2) for identification, by the following steps:

1×10⁵ Hela cells were cultured in a 6-well culture plate with 10% FBS DMEM medium. Next day (The cell number was 2×10⁵ and 70% cells were confluent), the liquid was discarded. 100 μl of the filtrate containing the adenovirus (about 10³ adenoviruses) prepared in (7) was added to the plate. After slightly shaken 3 times, the mixture was incubated in 5% CO₂ at 37° C. for 90 minutes.

The complement culture medium was added to the mixture to make the volume 1 ml. The cells were incubated in 5% CO₂ at 37° C. for 5-10 days till the complete CPE appeared. The cells were picked up and collected into a 1.5 ml EP puvette. The puvette was centrifuged and the supernatant was discarded. After being added 300 μl PBS, the puvette was frozen and thawed three cycles to release the adenovirus. The lysed solution was collected and centrifuged for 10 minutes. The supernatant of the lysed solution was collected and frozen at −80° C. DNA of the adenovirus was extracted with a Mini DNA isolation kit (Qiagen, Germany). A PCR was performed using the extracted adenovirus DNA as a template. Upstream primer used was 5′-CG GGA TCC GGG CCC CCA TTT CC-3′, and downstream primer used was 5′-TGC TCT AGA CAC AGG TGA TGT CG-3′. A 100 μl PCR mixture containing 10 ng of adenovirus DNA, 10 μl of 10×PCR buffer MgCl₂, 10 μl of 2 mM dNTP, 1 μl of 10 μM upstream primer, 1 μl of 10 μM downstream primer, 2.5 u HiFi Taq polymerase and water was prepared. The PCR conditions were set as follows: initial denaturation at 95° C. for 30 seconds, 95° C. for 45 seconds, 60° C. for 1 minute, 72° C. for 2 minutes, for a total 28 cycles, and final extension at 72° C. for 10 minutes. After conventional gel electrophoresis purification, a 1400 bp PCR product was obtained (primer for sequence assaying: 5′-AGCCGGAGCAGAGAGCCTTG-3′). Correct clones were selected for constructing Δ923-931ADV5/ASCHK1 and/or Δ923-931ADV5/ASPLK1 recombinant adenovirus.

### Example 3

### Construction of pCDNA3.1-ΔE3 Subclone Vector

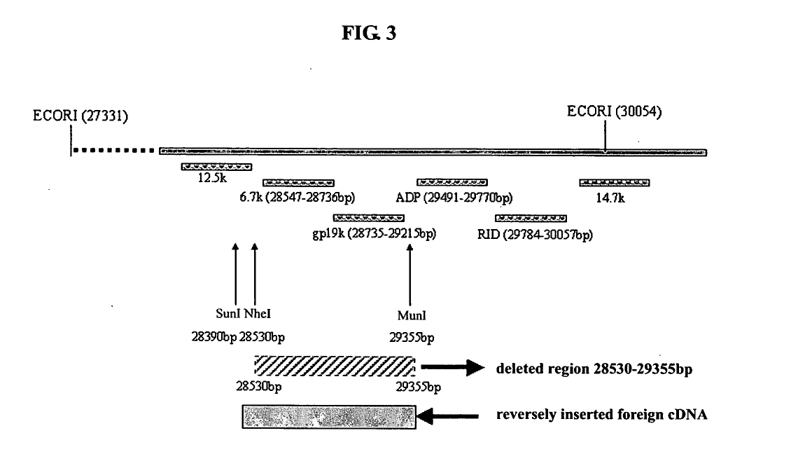

The whole name of ADV5 E3 is ADV5 early region 3 which encodes 7 proteins with an endogenous promoter. The sequence, structure and function of ADV5 E3 region are respectively as follows (FIG. 3):
12.5 k, 27858-28179 nt, function unclear;
6.7 k, 27547-28736 nt, function unclear;
gp19 k, 28735-29215 nt, binding MHC Class I-like antigen and inhibiting its presentation to the surface of cells, and escaping from CTL's elimination;
ADP, 29419-29770 nt, lysing cells and releasing the adenovirus;
RIDα, 29784-30057 nt, forming complex with RIDβ and preventing lysing of TNF and elimination of FAS antigen;
RIDβ, 30062-30458 nt; and
14.7 k, 30453-30837, inhibiting lysing of TNF.

The objective of the experiment is to delete a 28530-29355 nt sequence from E3 region for inserting a foreign therapeutical gene in E3 6.7 k/gp 19 k region of a recombinant adenovirus.

Deletion of a 28532-29360 nt of ADV5 E3 region by a 3-time PCR method Primers:

Primer 1: 5′-GGG TCA ACG GM TCC GCG CC-3′.

Primer 2: 5′-CCC ACA TAG AGT ATC GAT TGC GCC TTT GGC CTA ATA-3′, the underlined part being a complement to primer 3 indicated below and ATC GAT being a ClaI cleavage site.

Primer 3: 5′-GCC AAA GGC GCA ATC GAT ACT CTA TGT GGG ATA TGC TCC AG-3′, the underlined part being a complement to primer 2, and ATC GAT being a ClaI cleavage site;

Primer 4: 5′-GGG GM CM MC GCA GAT AGG-3′;
(1). Preparation of Fragment 1

DNA template of ADV5 adenovirus genome from a wild-type ADV5 (Chendu Benyuan Gene Therapy Company, Sichuan, China), was prepared as follows:

5×10⁶ 293 cells were seeded in a 75 cm² culture flask until next day (1×10⁷ cells, 70% cells being confluent).

3-4 hours before transfection, the cell culture medium was refreshed. To the 75 cm² culture flask from which the cell culture medium had been removed was added a mixture of 1 μl wild-type ADV5 adenovirus in 1 ml DMEM (MOI around 20).

The culture flask was shaken gently 3 times. Then the mixture was incubated in 5% CO₂ at 37° C. for 90 minutes.

To the mixture was added 9 ml 10% FBS DMEM. Then resultant mixture was incubated in 5% CO₂ at 37° C. for 72 hours. The culture medium was removed. The cells were washed with PBS 3 times. Remaining liquid was discarded. 800 μl of newly prepared cells lysis buffer was added. The resultant mixture was incubated at 37° C. for 1 hour. The lysed cell mixture was transferred into a 1.5 ml eppendorf, which was centrifuged at 4° C. at 12,000 g for 45 minutes.

The supernatant was collected, and then was extracted with phenol/chloroform with a same volume once. The upper layer of the extract was collected. 3M NaAc with 1/10 of the upper layer in volume, and anhydrous ethanol as 2 times of the upper layer in volume were added. The resultant mixture was incubated at −20° C. for 1 hour and then centrifuged at 12,000 g for 30 minutes. The precipitated adenovirus DNA was washed with 70% ethanol and resuspended with 25 μl TE. DNA concentration was determined in a spectrophotometer. The DNA was stored at −70° C. for later uses.

A 100 μl PCR reaction mixture containing ADV5 DNA as a template, 10 μl of 10×PCR containing MgCl₂, 10 μl of 2 mM dNTP, 1 μl of 10 μM primer 1, 1 μl of 10 μM primer 2, 200 ng/1 μl of ADV5 DNA, and 2.5 u of HiFi Taq polymerase was prepared.

The PCR conditions were set as follows: 94° C. for 30 seconds, then 28 cycles at 94° C. for 30 seconds, 62° C. for 1 minute and 72° C. for 1 minute, followed by 72° C. for 10 minutes. Fragment 1 (1,200 bp in length) was prepared after conventional gel electrophoresis purification.

(2). Preparation of Fragment 2

PCR conditions were set as described above except that primers 1 and 2 were replaced with primers 3 and 4, respectively. Fragment 2 (780 bp in length) was prepared after conventional gel electrophoresis purification.

(3). Preparation of Fragment 3

A PCR reaction was performed by mixing 50 ng of fragment 1 with 25 ng of fragment 2 as a template, in which primer 1 was used as the upstream primer, and primer 4 was used as the downstream primer. The PCR conditions were the same as the above. Fragment 3 (about 2,200 bp in length) was obtained after purification with a QIAquick 8 PCR Purification Kit. The PCR product was then digested with EcoRI overnight. The digested product was separated by electrophoresis in 1% agarose gel to extract the digested fragment for subsequent ligation reaction.

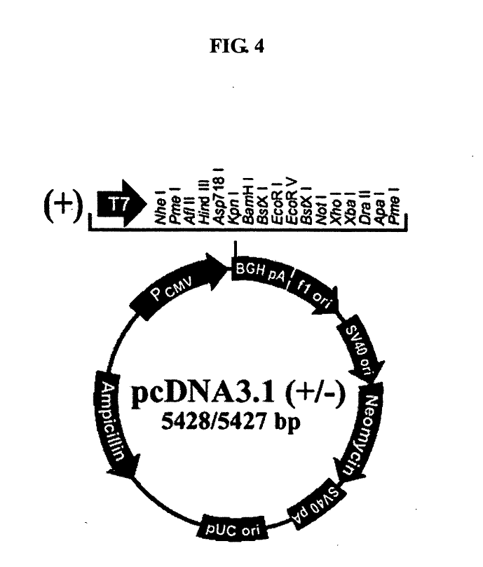

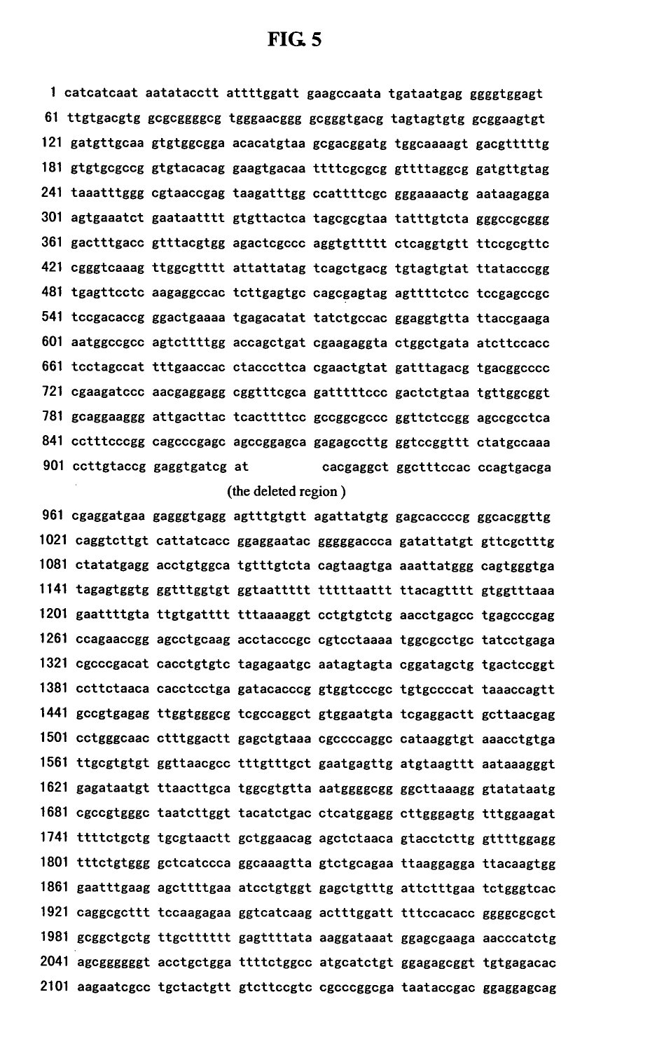

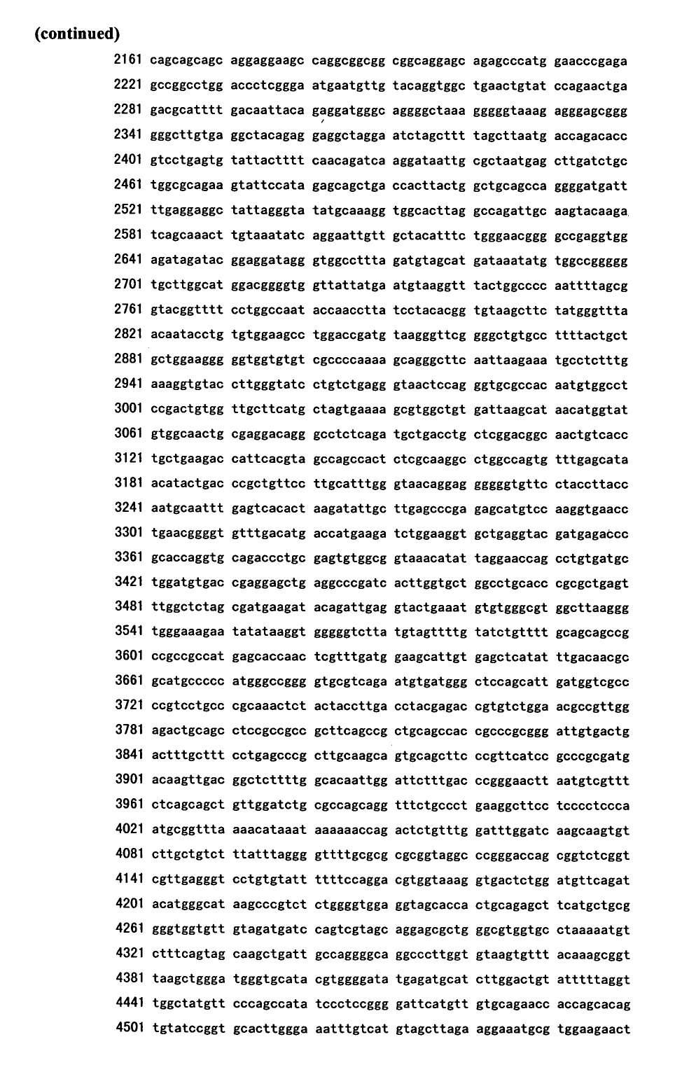

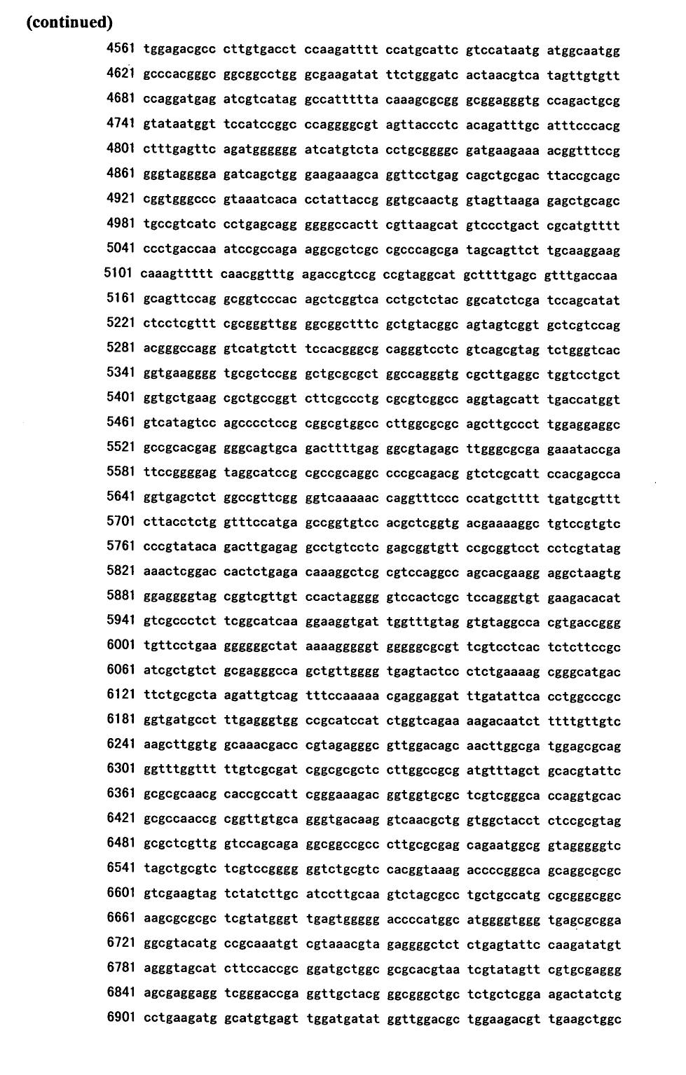

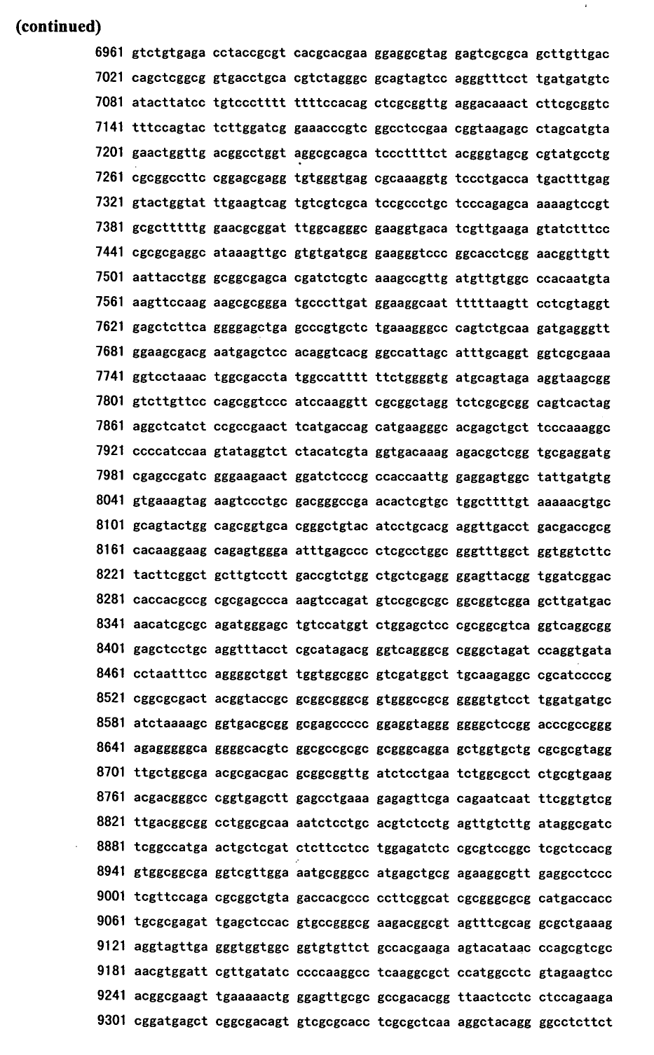

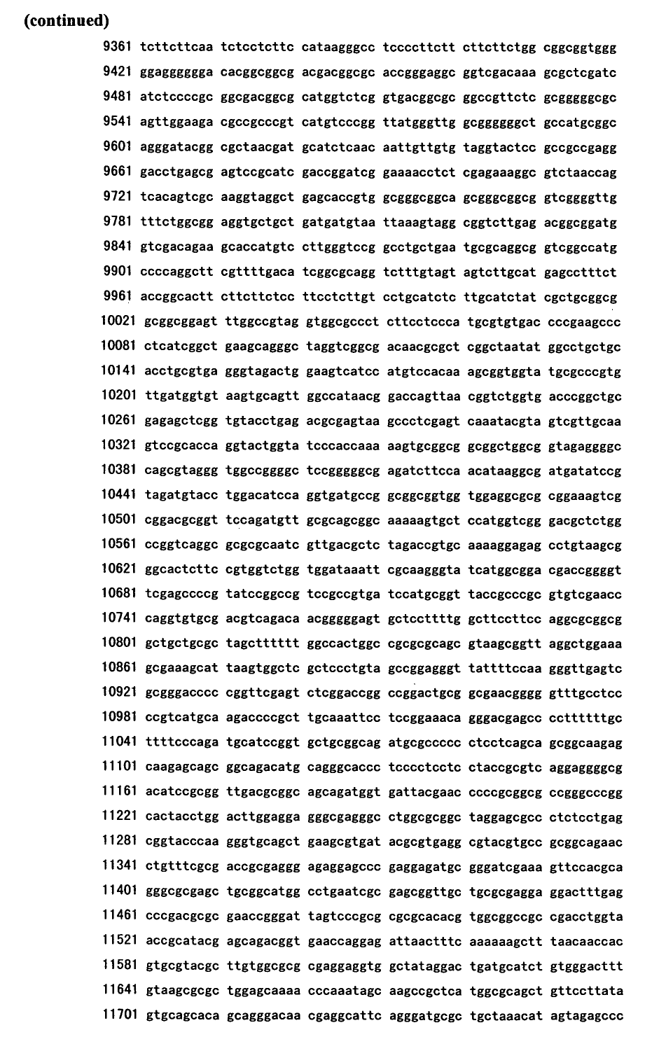

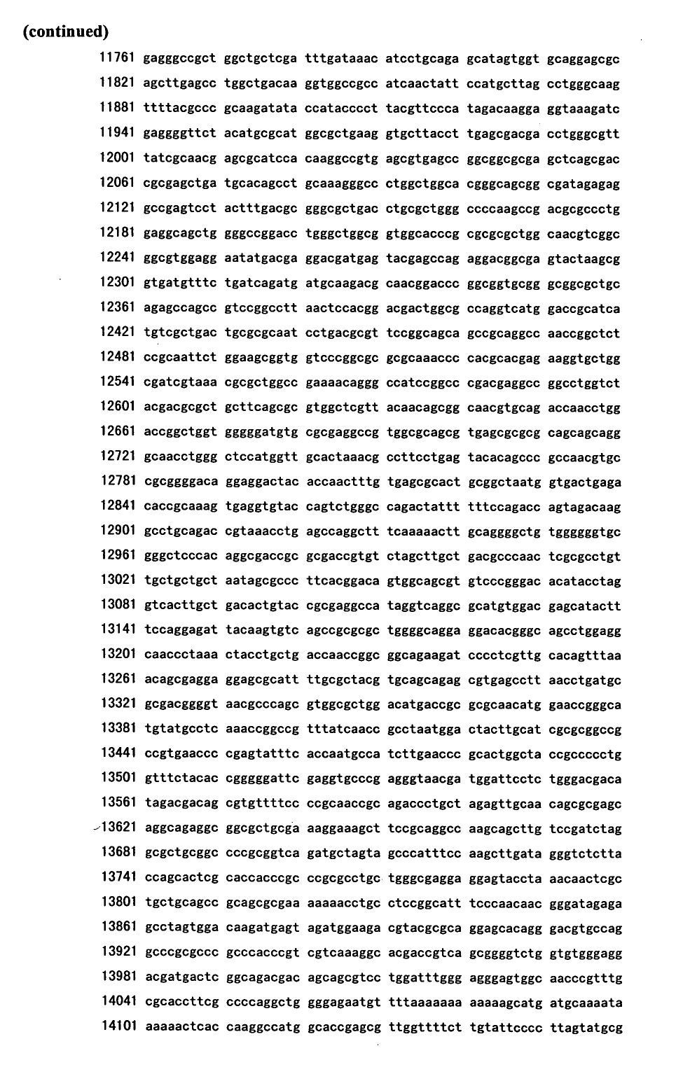

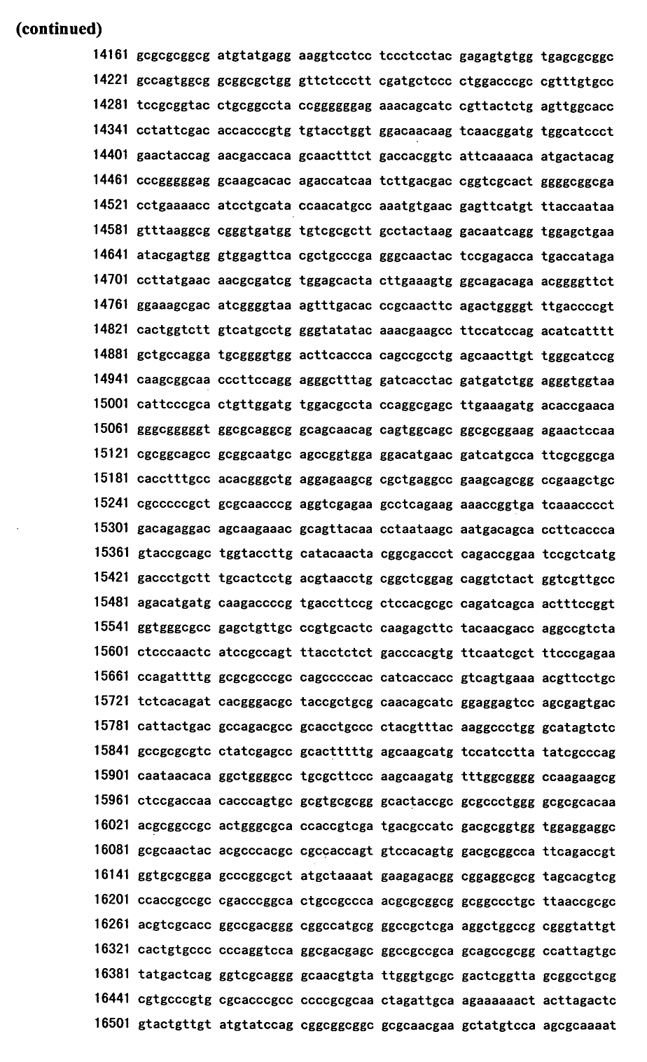

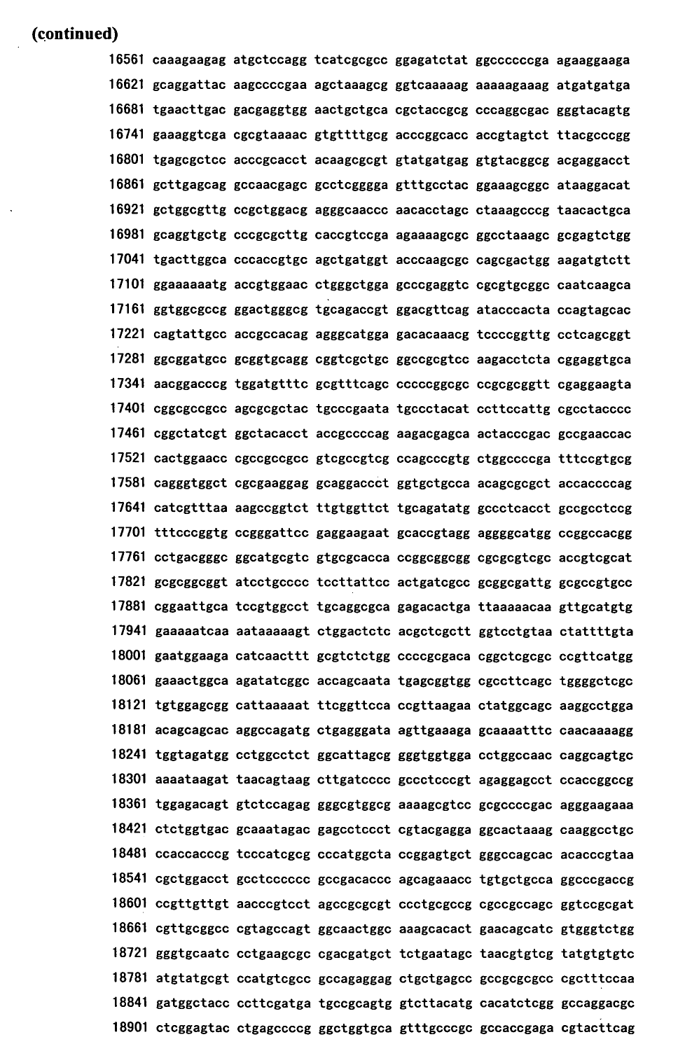

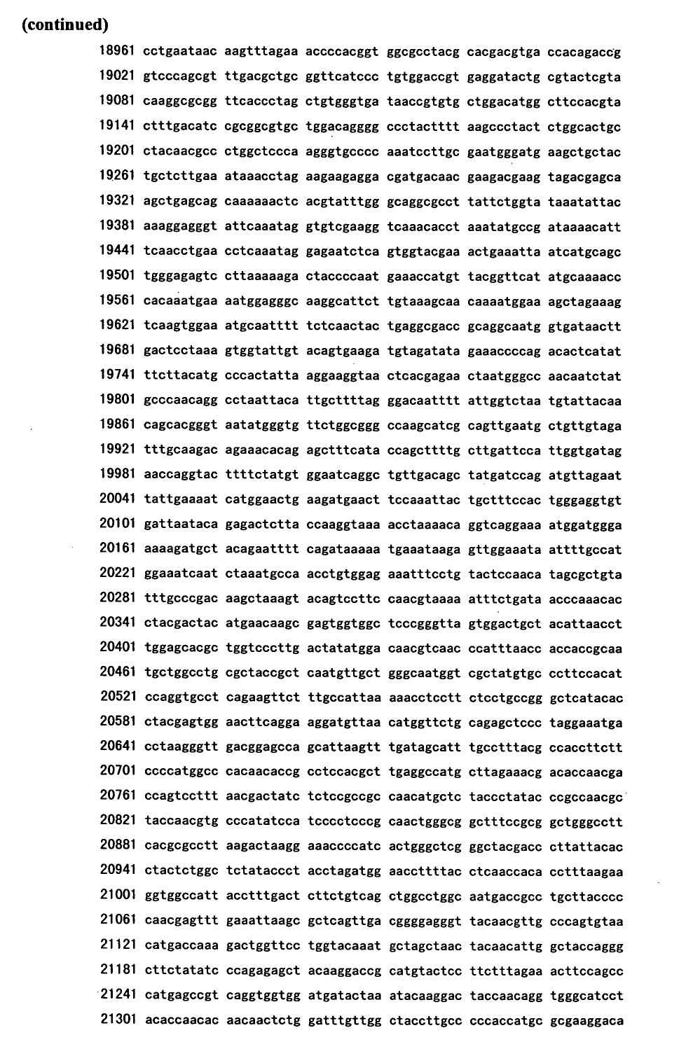

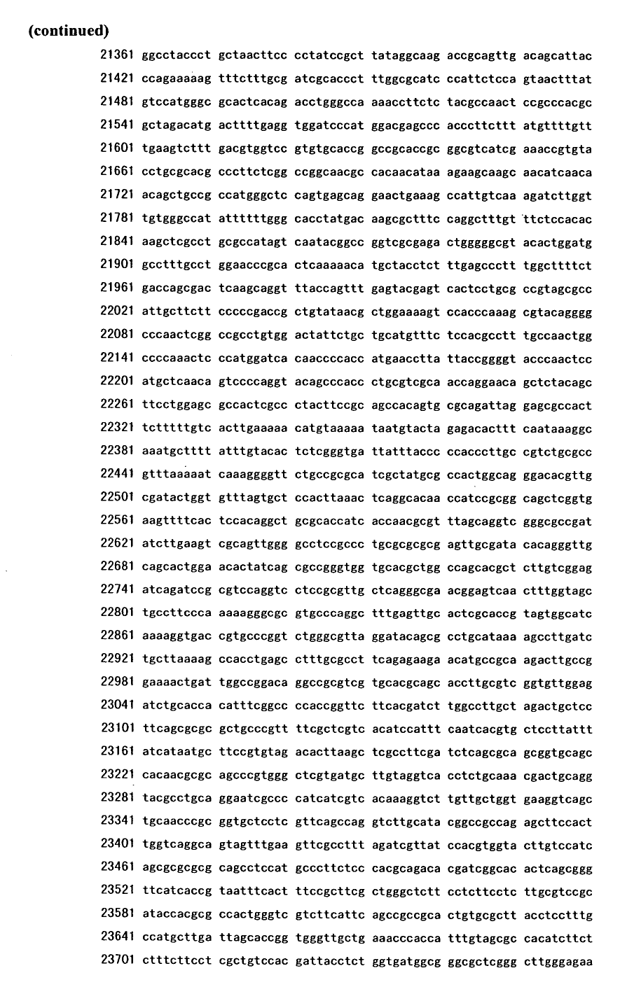

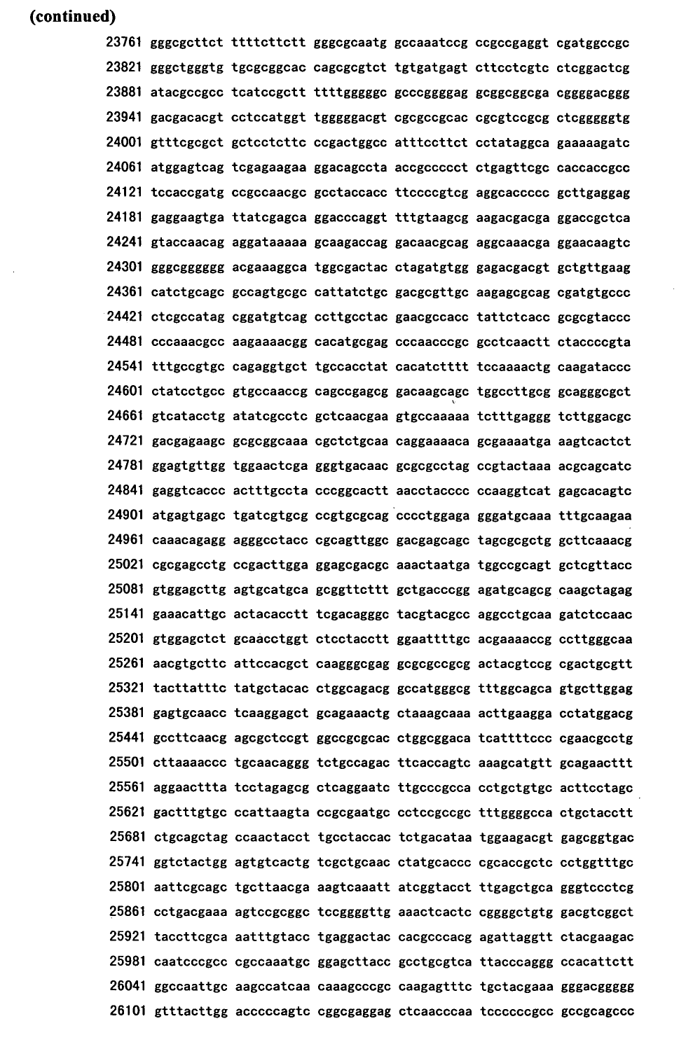

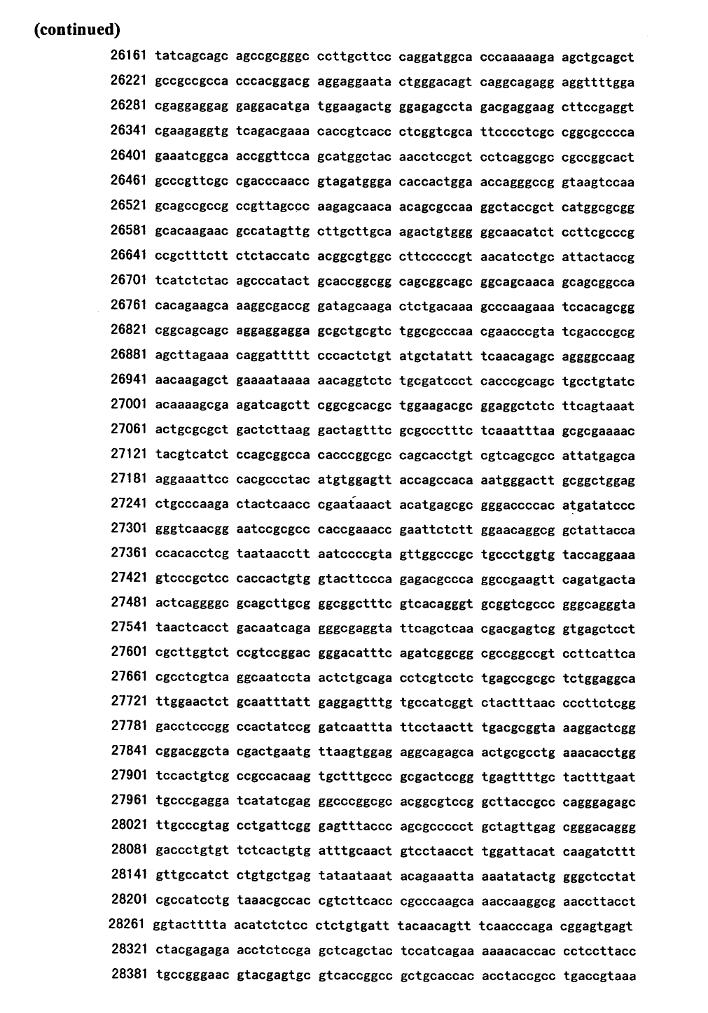

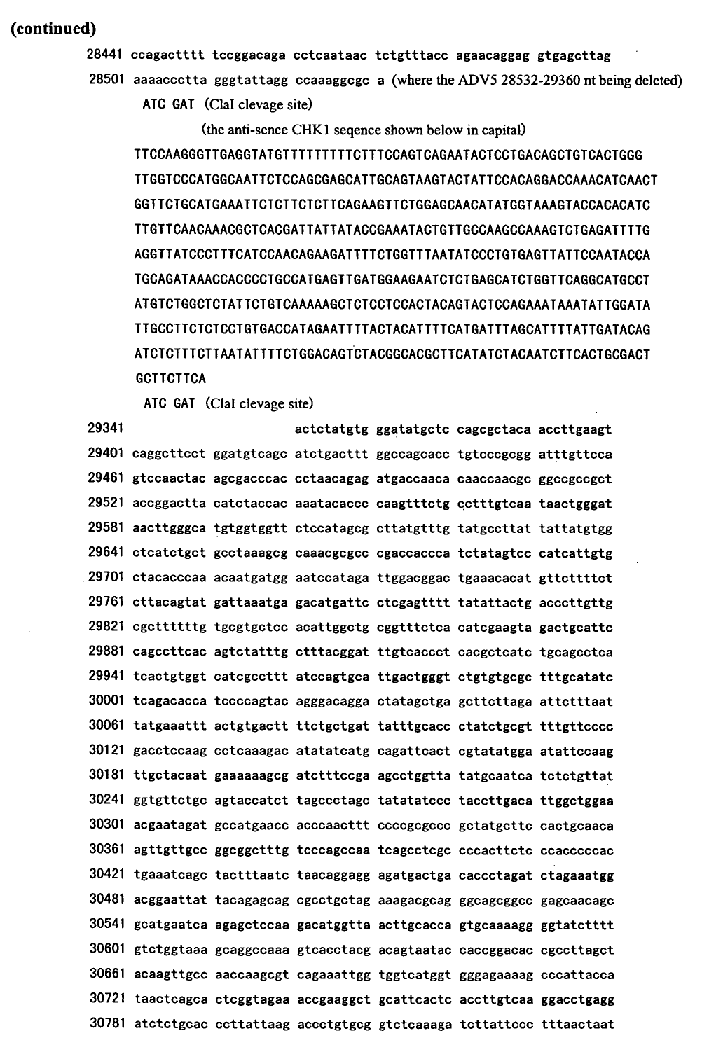

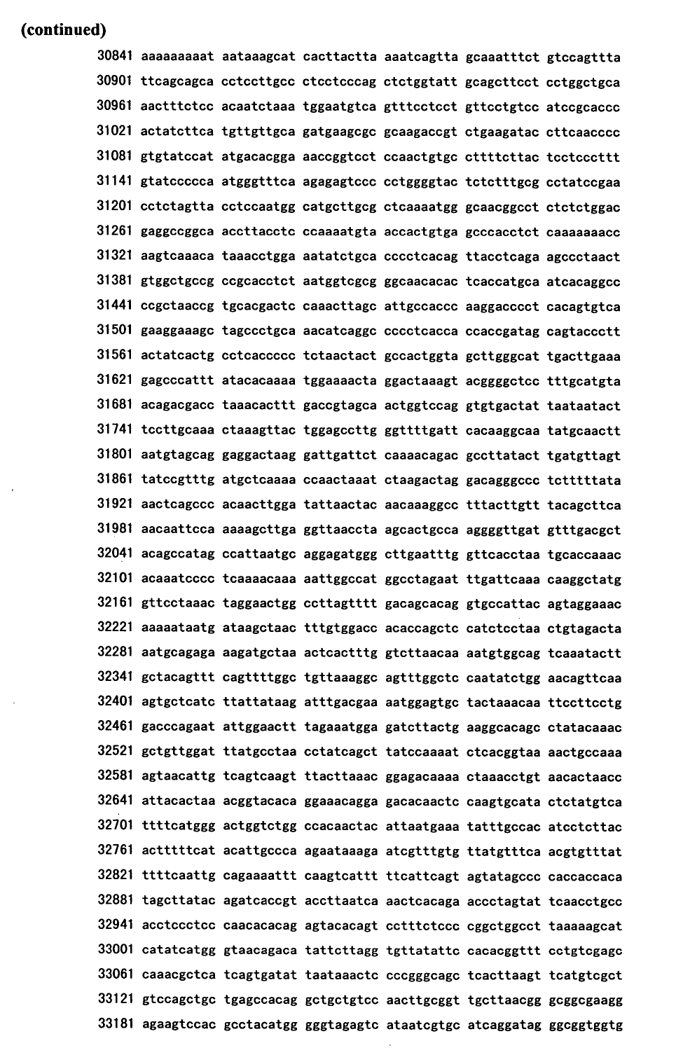

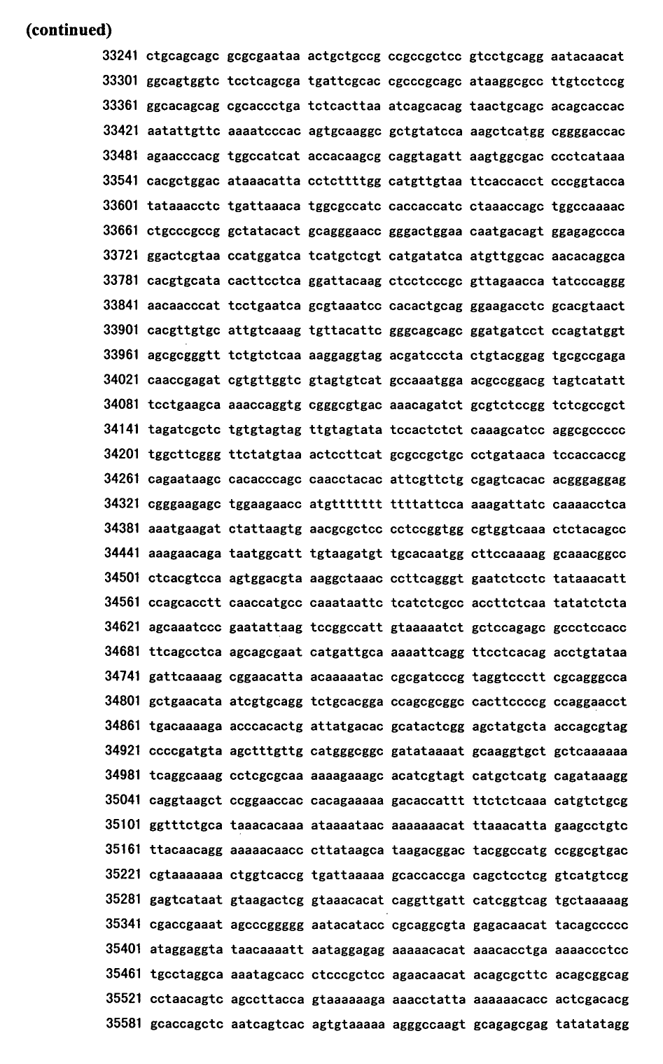

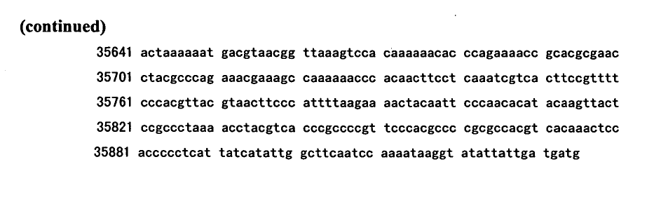

pCDNA3.1 (Invitrogen, USA, Cat: V79020) was digested with an EcoRI overnight. The digested product was separated by electrophoresis in 1% agarose gel. Then the digested product was recovered. After dephosphorylation, the product (The structure was shown in FIG. 4) was ligated with digested fragment 3. 1.5 μl of the ligated product was transformed into 100 μl of DH5α competent cells. Positive clones were selected. After micro-extraction of plasmids, pCDNA3.1 plasmid mutant pCDNA3.1-ΔE3 deleting the 28532-29360 nt sequence was obtained (identified by DNA sequencing assay).

### Example 4

### Inversely Inserting a Foreign CHK1 cDNA Fragment in Vector pCDNA3.1-ΔE3

Foreign CHK1 cDNA fragment was amplified from RNA of leukemia cell line HL-60 (ATCC, USA, Catalogue No. CCL-240). The RNA was extracted using RNeasy Mini Kit (QIAGEN, Germany, Catalogue No. 74104) in accordance with the description provided by the manufacturer for reverse transcription.

Reverse transcription condition: 4 μl of 5×MMLV buffer, 1 μl of 10 mM dNTP, 1 μl of OligdT (50 ng/ml), 2 μl of total RNA, 0.5 μl of Rnasin, 1 μl of MMLV reverse transcriptase and RNAase-free. The total volume was 20 μl.

Reverse transcription products were used for PCR. Upstream primer used was: 5′-CC ATC GAT CTG MG AAG CAG TCG CAG TG-3′ (the underlined part being a ClaI cleavage site); downstream primer used was: 5′-CC ATC GAT TTC CAA GGG TTG AGG TAT GT-3′ (the underlined part being a ClaI cleavage site); and the amplified fragment comprises a 853-250 nt sequence of CHK1 cDNA coding sequence with a total length of 620 bp.

A 100 μl PCR reaction mixture contains reverse transcription products as a DNA template, 10 μl of 10×PCR containing MgCl₂, 10 μl of 2 mM dNTP, 1 μl of 10 μM upstream primer, 1 μl of 10 μM downstream primer, 2 μl of reverse transcription product, 2.5 u of HiFi Taq polymerase was prepared.

The PCR conditions were set as follows: 94° C. for 30 seconds, then 30 cycles at 94° C. for 30 seconds, 62° C. for 1 minute and 72° C. for 1 minute, followed by 72° C. for 10 minutes.

After digestion with ClaI, the product was purified by conventional gel electrophoresis. Vector pCDNA3.1-ΔE3 was linealized with ClaI and separated by conventional gel electrophoresis. After dephosphorylation, the vector was ligated with the digested foreign CHKL cDNA fragment. 1.5 μl of the ligation product was transformed into 100 μl of DH5α competent cells. Positive clones were selected, and micro-extraction of the plasmid was performed. After a DNA-sequencing identification, a plasmid mutant, pCDNA3.1-ΔE3/ASCHK1, in which the foreign CHK1 cDNA fragment was inversely inserted at the ClaI cleavage site, was obtained.

### Example 5

### Inversely Inserting a Foreign PLK1 cDNA Fragment into Vector pCDNA3.1-ΔE3

Foreign PLK1 cDNA fragment was amplified from RNA of leukemia cell line HL-60 (ATCC, USA, Catalogue No. CCL-240). The RNA was extracted using RNeasy Mini Kit (QIAGEN, Germany, Catalogue No. 74104) in accordance with the description provided by the manufacturer for reverse transcription.

Reverse transcription condition: 4 μl of 5×MMLV buffer, 1 μl of 10 mM dNTP, 1 μl of OligdT (50 ng/ml), 2 μl of total RNA, 0.5 μl of Rnasin, 1 μl of MMLV reverse transcriptase and RNAase-free. The total volume was 20 μl.

Reverse transcription products were used for PCR. Upstream primer used was: 5′-CC ATC GAT GGC TCC ACC GGC GAA AGA GA-3′ (the underlined part being a ClaI cleavage site); downstream primer used was: 5′-CC ATC GAT GCA GCT CGT TAA TGG TTG GG-3′ (the underlined part was a ClaI cleavage site); and the amplified fragment comprises a 960-161 nt sequence of PLK1 cDNA coding sequence with a total length of 820 bp.

A 100 μl PCR reaction mixture containing the reverse transcription product as a DNA template, 10 μl of 10×PCR containing MgCl₂, 10 μl of 2 mM dNTP, 1 μl of 10 μM upstream primer, 1 μl of 10 μM downstream primer, 2 μl of reverse transcription product, 2.5 u of HiFi Taq polymerase was prepared.

The PCR conditions were set as follows: 94° C. for 30 seconds, then 30 cycles at 94° C. for 30 seconds, 62° C. for 1 minute and 72° C. for 1 minute, followed by 72° C. for 10 minutes.

An 850 bp PCR product was obtained. After digestion with ClaI, the PCR product was purified by conventional gel electrophoresis. Vector pCDNA3.1-ΔE3 was linealized with ClaI and separated by conventional gel electrophoresis. After dephosphorylation, the vector was ligated with the digested foreign PLK1 cDNA fragment. 1.5 μl of the ligation product was transformed into 100 μl of DH5α competent cells. Positive clones were selected and mini-extraction of plasmids was performed. After a DNA-sequencing identification, a plasmid mutant, pCDNA3.1-ΔE3/ASPLK1, in which the foreign PLK1 cDNA fragment sequence was inversely inserted at the ClaI cleavage site, was obtained.

### Example 6

### Δ923-931ADV5/ASCHK1 Recombinant Adenovirus

The preparation of recombinant adenovirus was performed with reference to the ligation protocol described in “Adenovirus Methods and Protocol”, edited by William S. M. Wold, Construction of Mutations in the Adenovirus Early Region 3<E3> Transcription Units, p11-24.

1. Extraction of TP DNA

(1). 2×10⁸ Δ923-931ADV5 recombinant adenovirus vector was dissolved in a 10 ml DMEM, the MOI of which was about 10. 293 cells were seeded in a 75 cm² culture flask with 80-90% cells confluent. The cell number was about 2×10⁷. 1 ml of the DMEM solution was added to the flask and the flask was shaken gently 3 times. Then the mixture was incubated in 5% CO₂ at 37° C. for 90 minutes. 10% FBS DMEM was added to the mixture to make the final volume 10 ml. The mixture was incubated in 5% CO₂ at 37° C. for 4-5 days, till the cell number was 2×10¹⁰. The supernatant was removed. 10 ml of 10% FBS DMEM was added to perform 3 cycles of freezing and thawing to release the adenovirus, the density of which was 2×10⁹/ml. The solution was divided into 10 tubes (each 1.0 ml) for freezing storage. The second amplification was performed. Before transfection, 9 ml of DMEM was added to a tube of freezing stored solution to the final concentration of 2×10⁸/ml. The cells in the 10×75 cm² culture flasks were transfected. 1 ml of the solution was added into 75 cm² flasks (10) containing about 2×10⁷ 293 cells. These flasks were shaken gently 3 times and incubated in 5% CO₂ at 37° C. for 90 minutes. 10% DMEM was added to the flasks to the final volume of 10 ml and incubated in 5% CO₂ at 37° C. for 4-5 days. Then the mixture was centrifuged and the supernatant was discarded. The transfection process was repeated 10 times. The total adenoviruses obtained were about 2×10¹³, and was stored at −80° C. for later purification.

(2). Collection, Condensation and Freezing Storage of 293 Cells

The 293 cells were re-suspended with 30 ml of PBS. The cell suspension was aliquoted into two 50 ml centrifugal tubes which were in turn frozen in liquid nitrogen, and then thawed in a water bath at 37° C. 3 times to rupture and lyse the cells, thereby releasing Δ923-931ADV5. The cell debris was removed with a common centrifuge at 2,500 rpm for 5 minutes. The supernatant was collected and frozen at −70° C. for later purification.

(3). Separation and Purification of Δ923-931ADV5

Δ923-931ADV5 was purified with CsCl₂ density gradient ultracentrifugation.

The frozen supernatant was added to a CsCl₂ solution (Gibco, USA, Catalogue No.456-32). It was centrifuged at 35,000 rpm (150,000×g) at 4° C. in two SW-40TL rotors for 1 hour.

Preparation of CsCl₂ Gradient Centrifugal Solution:

Solution A (1.5 gm/ml CsCl₂ gradient centrifugal solution): 30 g CsCl₂ was dissolved in a PBS till a 42.5 ml final volume;
Solution B (1.35 gm/ml CsCl₂ gradient centrifugal solution): To 15 ml of Solution A was added 7 ml of PBS till a 22 ml final volume; and
Solution C (1.25 gm/ml CsCl₂ gradient centrifugal solution): To 11 ml of Solution A was added 9 ml of PBS till a 20 ml final volume.

After filtration and sterilization, the CsCl₂ gradient centrifugal solution was prepared.

To a 12 volumes centrifugal tube were added in turn: 0.5 ml of Solution A, 3.0 ml of Solution B, and 3.0 ml of Solution C.

After forming gradient, 6 ml of the lysed cell supernatant was added to each tube. The weight of each tube was adjusted with PBS.

After centrifugation, adenoviruses appeared as a white band located between Solution B and Solution C. The mid layer containing adenovirus was removed with a Pasteur's sucker to a 100 ml sterilized centrifugal tube after the upper protein layer was removed.

The resultant adenovirus solution was diluted to 72 ml with Solution B, which was aliquoted into 6 sterilized centrifugal tubes. After balancing the weight, the tubes were centrifuged for 18 hours.

A white band containing Δ923-931ADV5 adenovirus was formed. The mid layer containing adenovirus (around 15 ml) was removed with a Pasteur sucker to a 100 ml sterilized centrifugal tube after the upper protein layer was removed.

(4). Removal of Trace CsCl₂ and Other Small Molecular Impurities with Dialysis Method
Dialysis solution: 10 mmol Tris-HCl pH7.5, 1 mmol MgCl₂; Dialysis Volume: 1000 ml×3.
   Dialysis Method:

A magnetic stick was added into the dialysis solution and the solution was pre-cooled at 4° C., and kept in a magnetic stirrer in dark at 4° C. overnight. The dialysis was performed 2 times under the same conditions and the adenovirus obtained was purified adenovirus.

(5). Purification of Δ923-931ADV5 Adenovirus with TP DNA by Column Chromatography

A chromatographic column (Hubei Science Apparatus Co., Catalogue No. SSM-213) was loaded with 250 ml of Sepharose 4B (Sigma, USA, Catalogue No. 4B-200) at 4° C. The speed of flow was set at 18 ml/h. To the adenovirus solution was added a solution of 8M guanidine thiocyanate hydrochloride containing 2 mmol/l proteinase inhibitor (PI) pefabloc (Boehringer Mannheim, Catalogue No.1 429 876) having the same volume as the adenovirus solution. After the mixture was cooled with ice for 10 minutes, it was loaded into the chromatography column. TP-DNA was collected (A260/280=1.8-2.0). The DNA concentration was determined, and the DNA was stored at 4° C. for later use.

2. Ligation Reaction

Plasmid pCDNA3.1-ΔE3/ASCHK1 or pCDNA3.1-ΔE3/ASPLK1 was digested with EcoRI. 10 μg of EcoRI-digested fragments inserting DNA fragment was dephosphorylated and separated by electrophoresis in 1% agarose gel for ligation reaction.

To 2.5 μg of Δ923-931ADV5 TP DNA, after digested with EcoRI, were added 5 μg of EcoRI-digested fragments inserting DNA fragment and 10U T4 ligase. The ligation reaction was performed at 16° C. to obtain a ligation product.

3. Co-Infecting 293 Cells with the Ligation Product

(1). 5×10⁵ 293 cells were seeded in a 60 mm culture plate with 10% FBS DMEM medium. The cell number reached 1×10⁶ next day, 70% of which were confluent. 3-4 days before transfection, the culture medium was refreshed.

(2). The ligation product was mixed with 20 μg of salmon sperm DNA. Then the sterilized water was added until the final volume reached 450 μl. 50 μl of 2.5M CaCl₂ solution was added thereto. The resultant DNA/CaCl₂ mixture was slowly added to 500 μl of sterilized 2×HBS (280 mM NaCl, 43 mM HEPES, 10 mM KCl, 10 mM Na₂HPO₄.7H₂O, 2% dextrose, pH7.05-7.15). The reaction was performed for 45-60 minutes (slightly turbid precipitation being formed).

(3). 500 μl of the mixture acquired from step (2) was added to the 293 cells in the 60 mm culture plate (step (1)). Then the mixture was incubated in 5% CO₂ at 37° C. for 1/2 hour. The liquid was removed. After washed with PBS, the cells were treated with 15% glycerol/DMEM for 1-2 hours. Then a complement culture medium was added after washed with PBS.

(4). The complement culture medium was removed. To the mixture was added 5 ml of newly prepared 10% low melting point agarose PBS (prior to use, it being melted with boiling water and kept at 45° C., then mixed with 30 ml 10% FBS DMEM to make the final concentration 1.2%). Another 3 ml of the newly prepared 10% low melting point agarose PBS was added every 4-5 days.

(5). After 14-21 days, plaques appeared. 6-12 plaques were selected and marked. 200 μl of pipettor tips were used to transfer the plaques to a 0.5 ml 24-well plate containing 10% FBS DMEM, the plaques were sieved after being at 37° C. for 24 hours in the plate to obtain adenovirus filtrate.

(6). 100 μl of the filtrate (about 10³ adenoviruses) was added to a 24-well culture plate each containing a newly cultured 293 cells (incubated in 10% FBS DMEM medium one day). The plate was shaken gently 3 times. The cells were incubated in 5% CO₂ at 37° C. for 90 minutes. Recombinant adenovirus constructs Δ923-931ADV5/ASCHK1 and Δ923-931ADV5/ASPLK1 were obtained.

4. Identification of Recombinant Adenovirus Construct

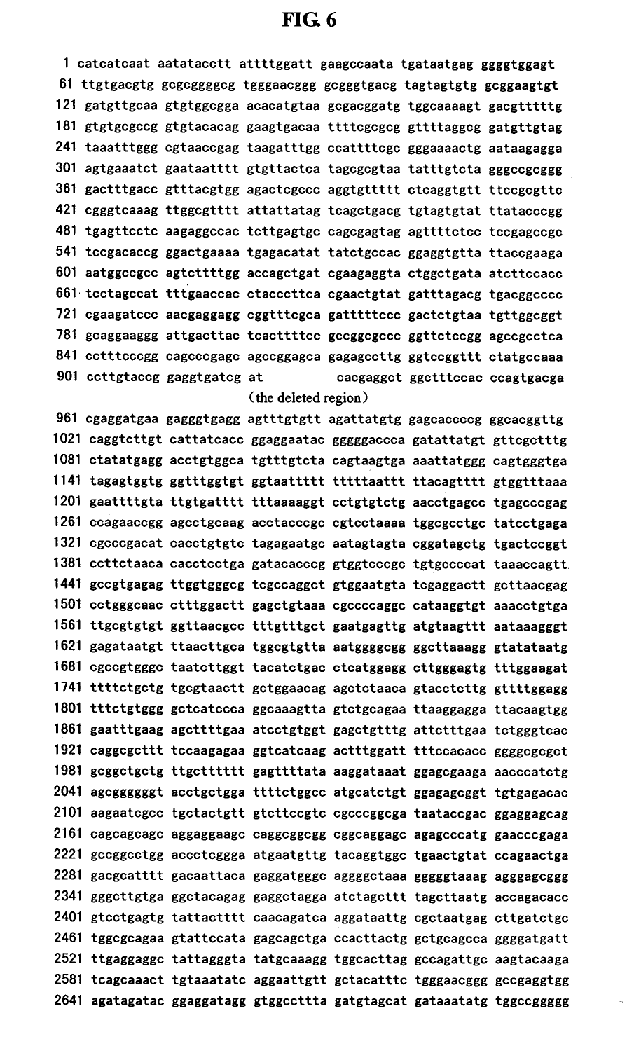

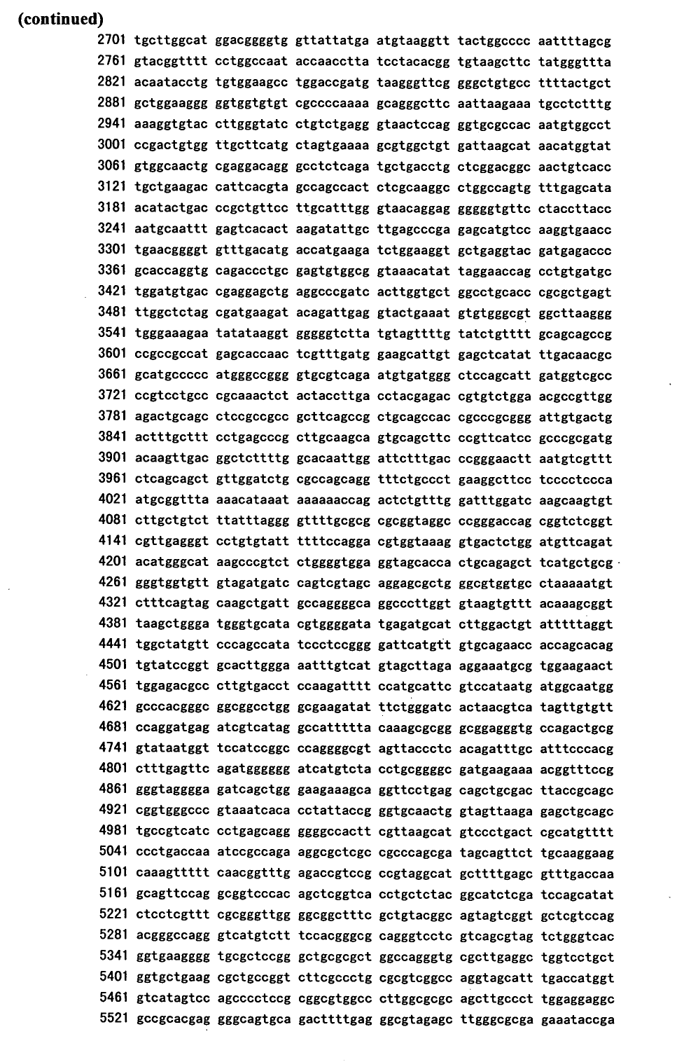

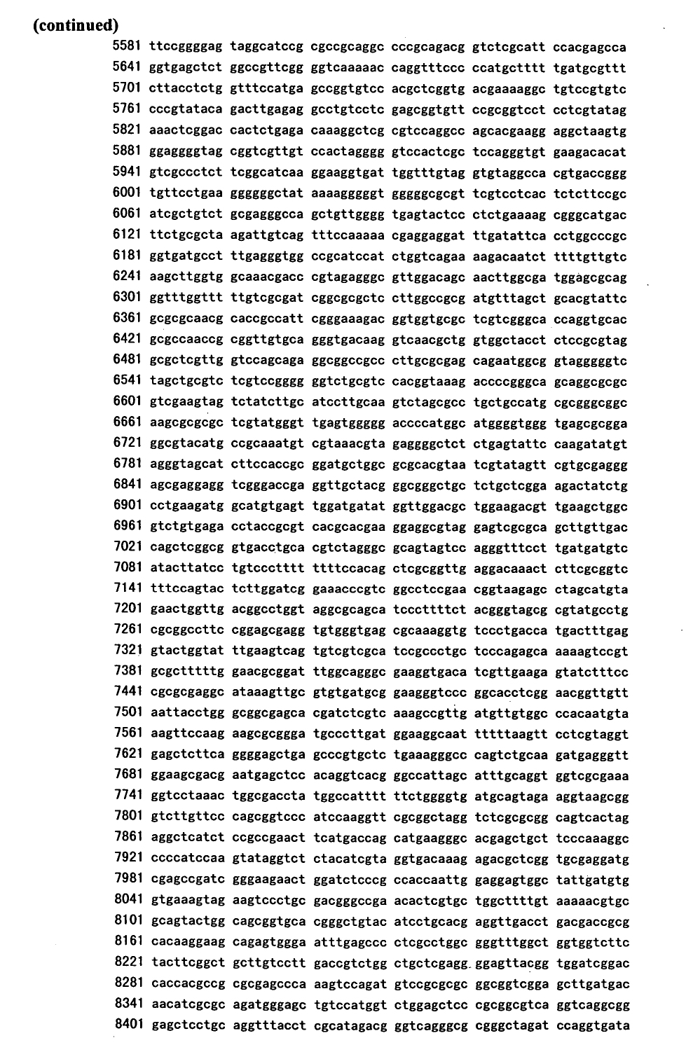

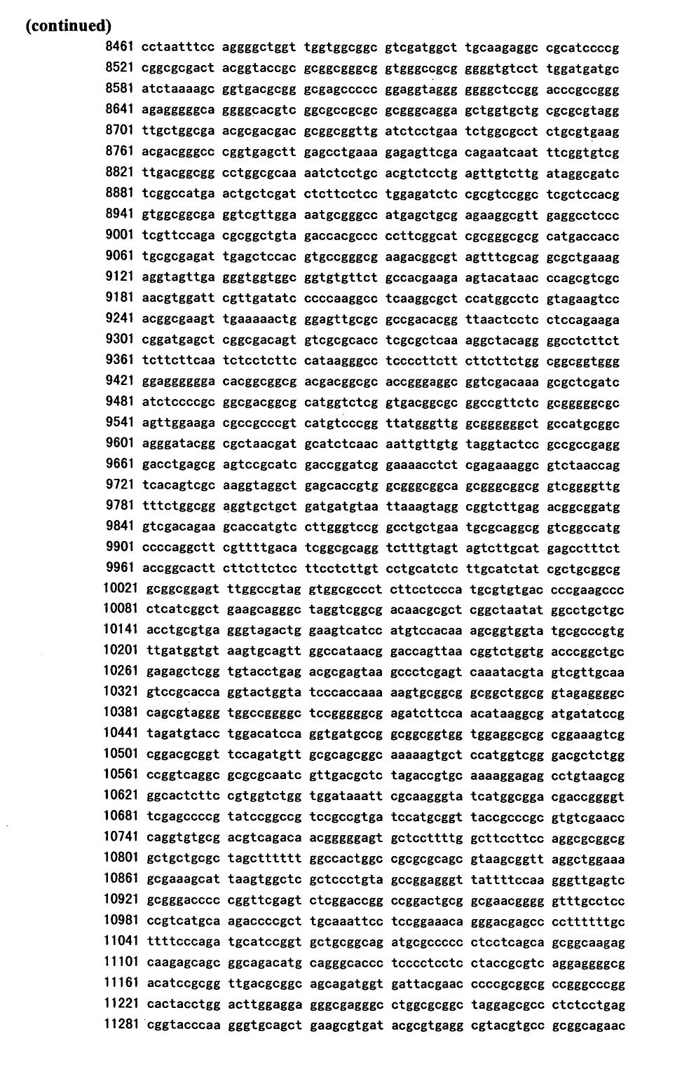

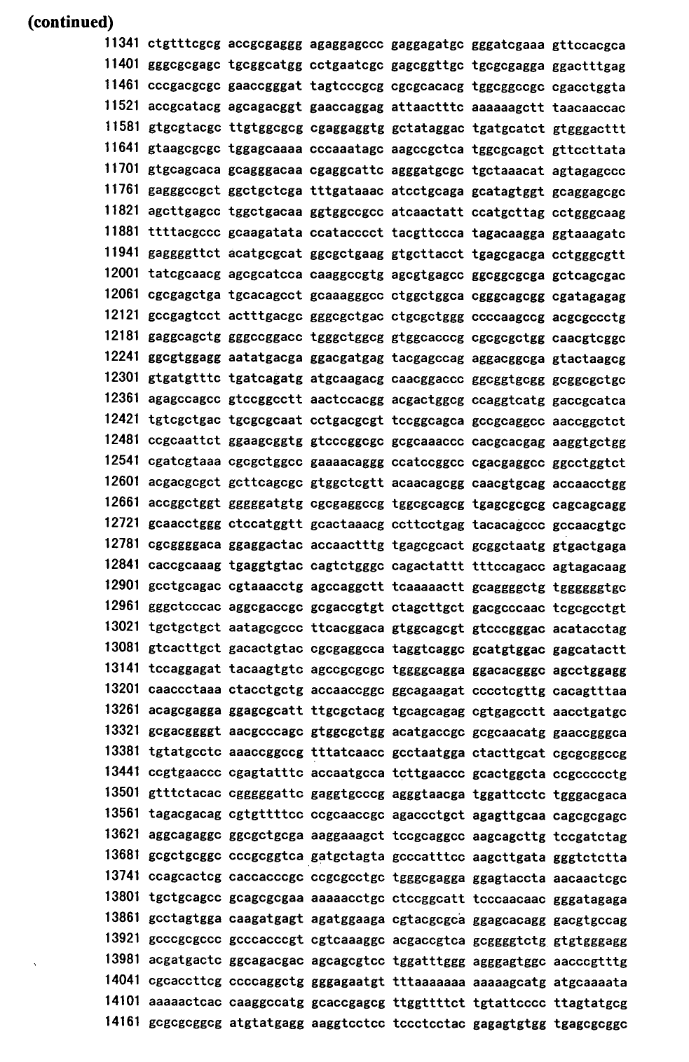

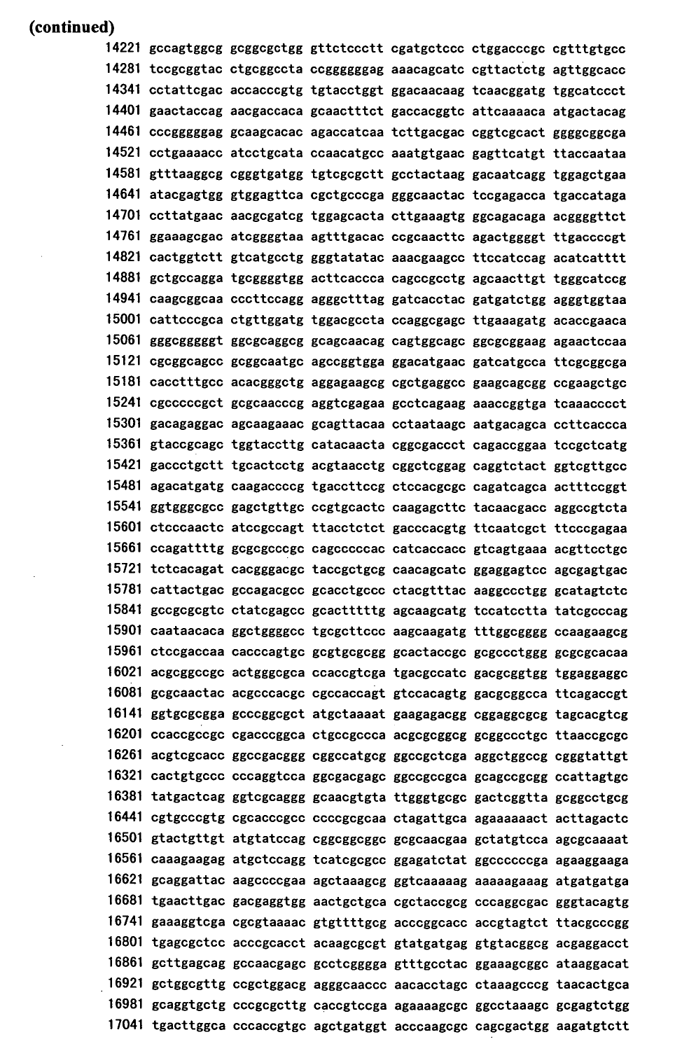

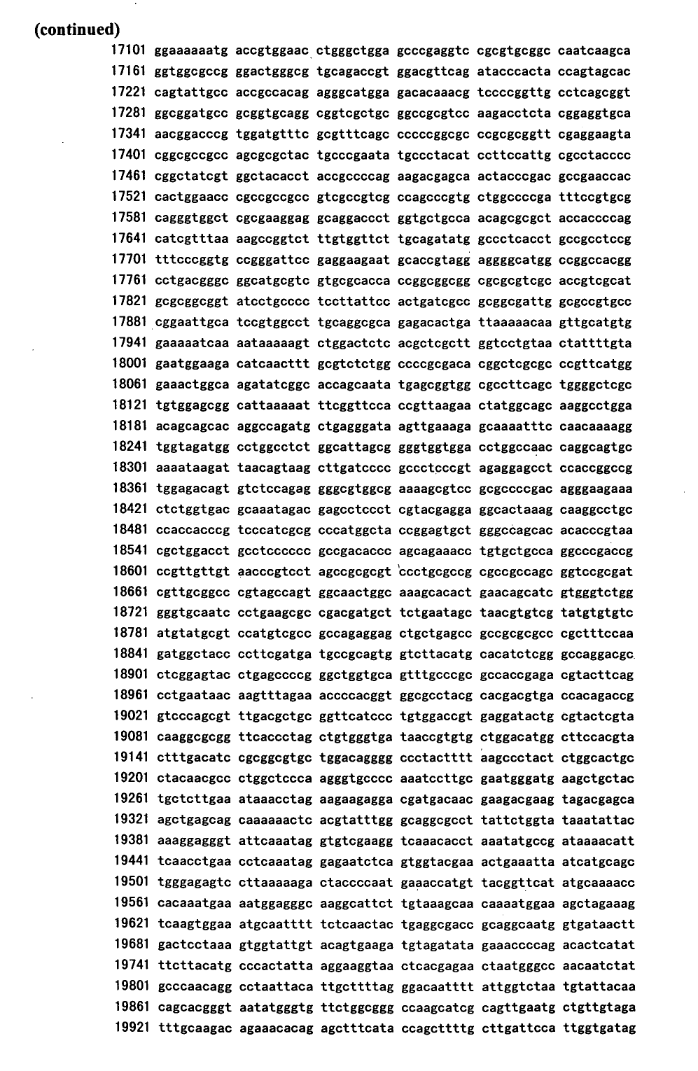

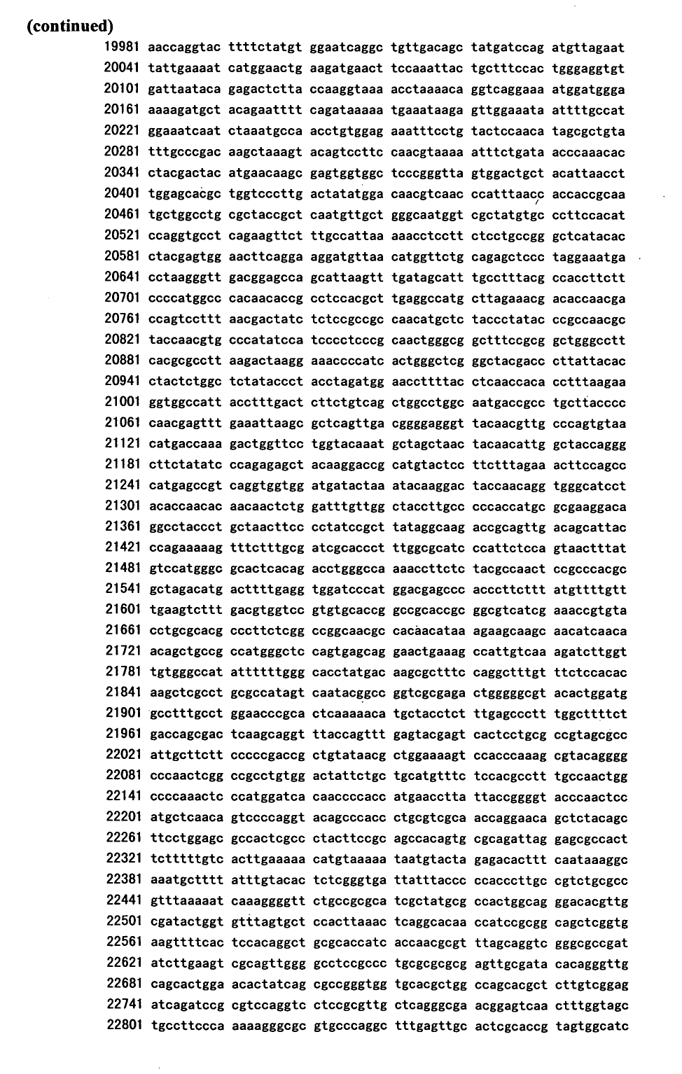

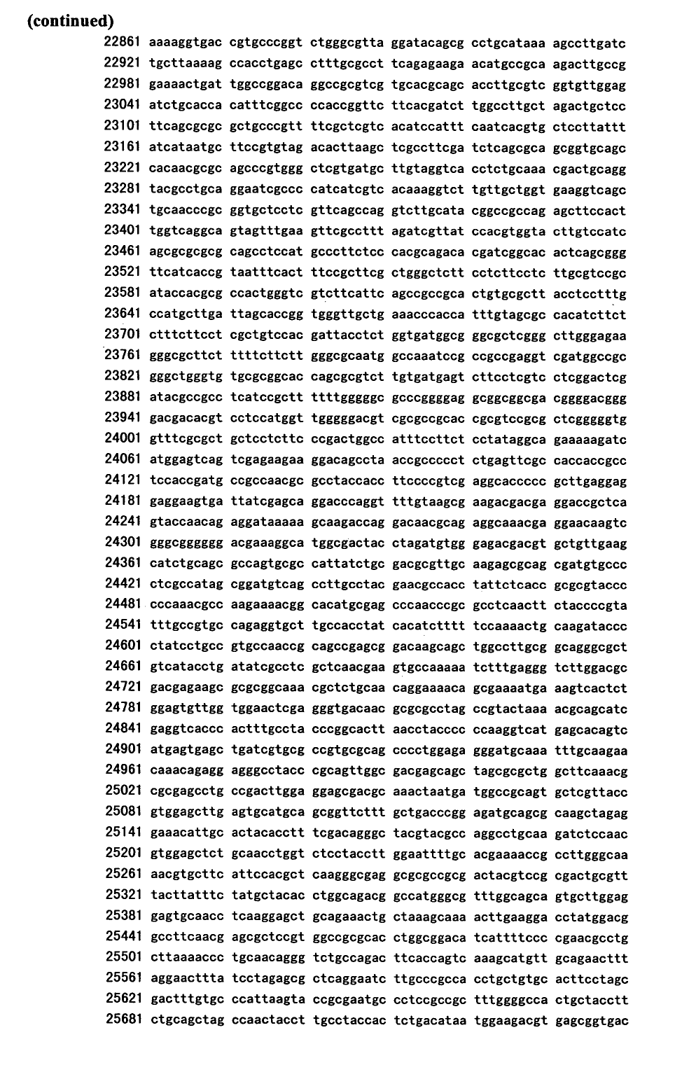

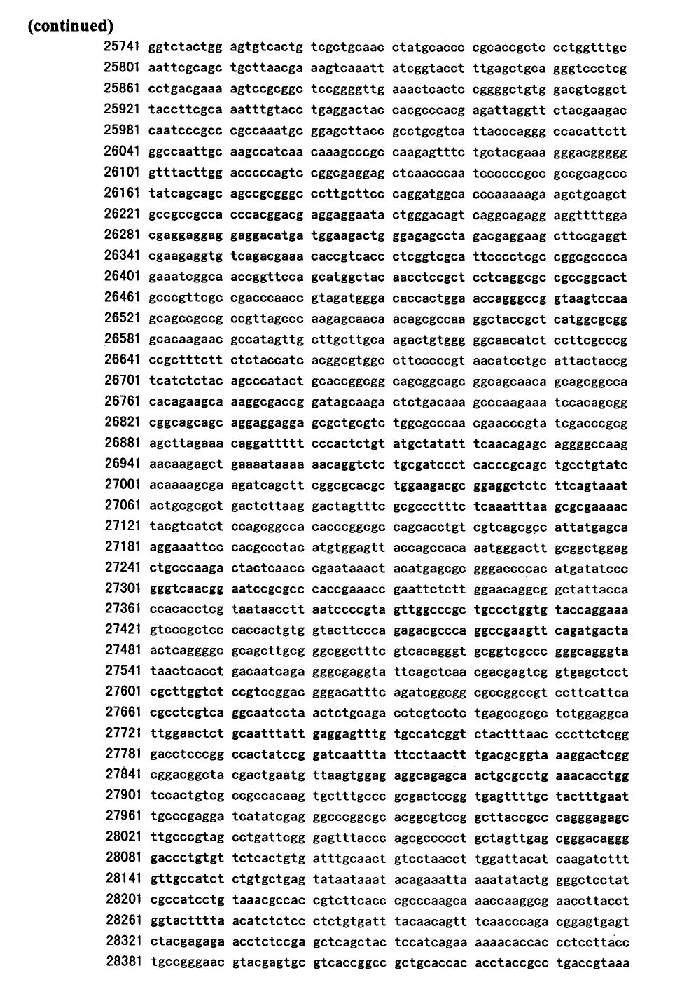

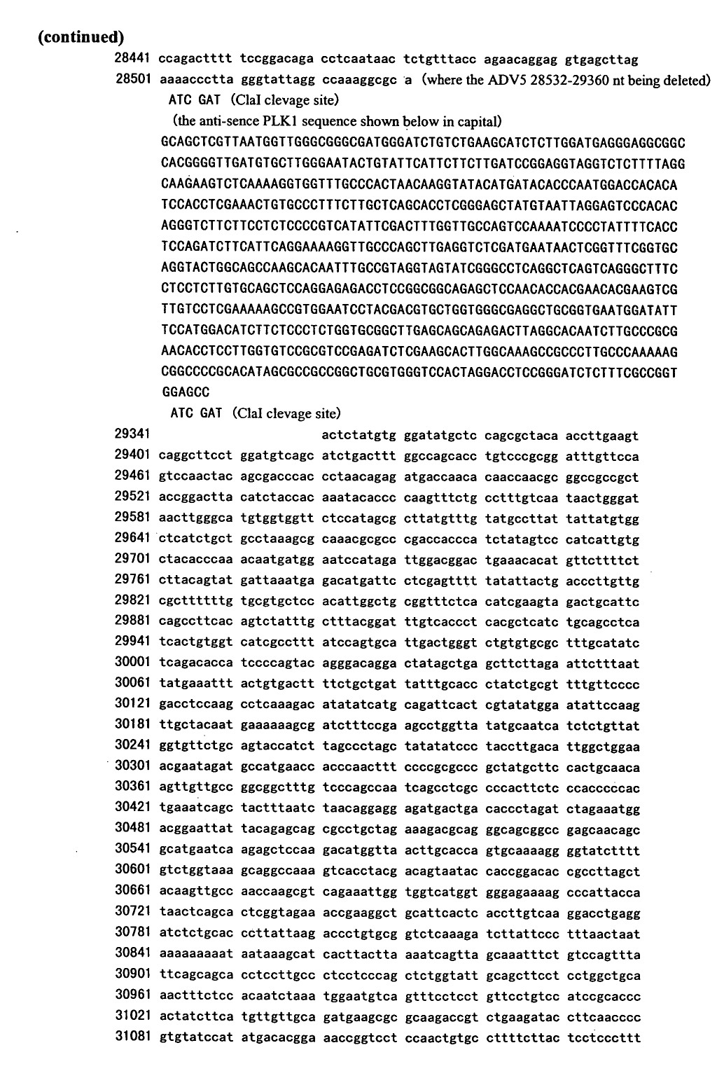

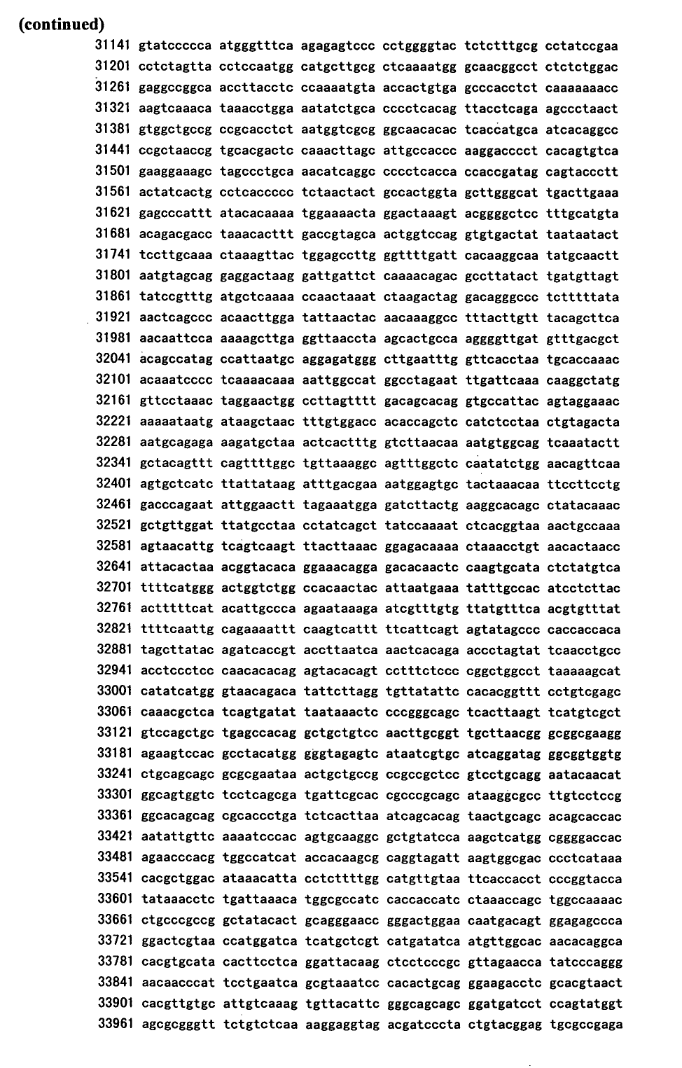

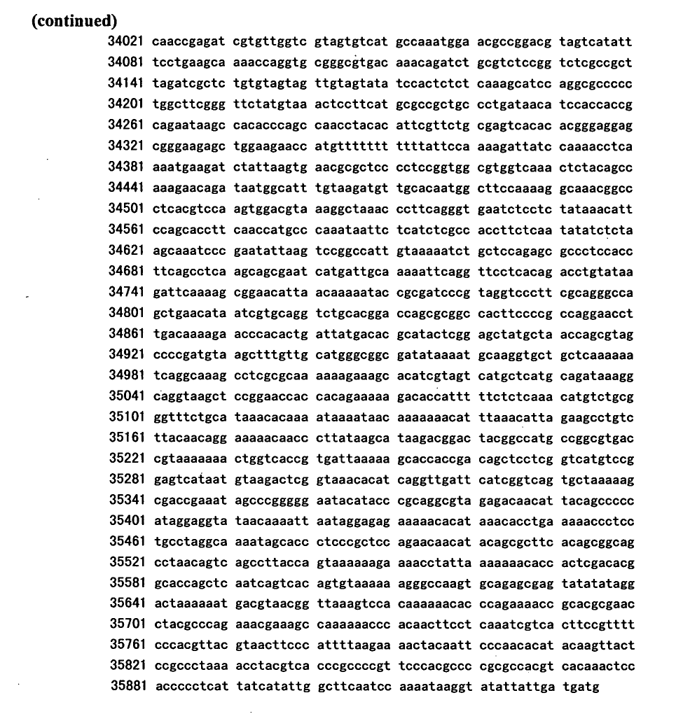

The adenovirus DNA was extracted from the recombinant adenovirus constructs acquired from the above and sequenced for identification. FIG. 5 and FIG. 6 respectively showed the sequence of Δ923-931ADV5/ASCHK1 and Δ923-931ADV5/ASPLK1. From the Figures, the ADV5 923-931 nt sequence (CTT ACC TGC) and 28532-29360 nt of ADV5 E3 region comprising a foreign CHK1 cDNA fragment (corresponding to 853-250 nt of CHK1 mRNA) or PLK1 cDNA fragment (corresponding to 960-161 nt of PLK1 mRNA) were deleted. The other sequences of the constructs were identical to those of wild-type ADV5.

### Example 7

### Bioactivity Assaying

1. Identification of In Vitro Tumor-Specific Replication of Recombinant Adenovirus Constructs

The objective of the present experiment is to identify the replication characteristics of recombinant adenovirus construct in a serial of tumor and normal cells. Wild type ADV5 was used as a positive control and ADV-TK (a replication-deficient adenovirus constructed by the inventors) was used as a negative control. Cell lines were selected from A549 (ATCC, USA, Catalogue No. CCL-185), Hela, human osteogenesis blastoma U-2OS cell line (P53+, RB−; ATCC, USA, Catalogue No. HTB-96), human metastatic adenoma HS700T cell line (P53−, RB−; ATCC, USA, Catalogue No. HTB-147) and human colon cancer MCF-7 cell line (P53+, RB+; ATCC, USA, Catalogue No HTB-22). Because the cell lines selected possess different p53 and RB phenotypes and bear different tissue origins, the experiment can show the representative of different types of tumors. Normal tissue cells including primary vascular endothelial cells, primary lung tracheole endothelial cells, prostate endothelial cells and bone marrow mononuclear cells separated from clinical samples were selected to use in the experiment. The selected cells can mostly contact the adenovirus administrated intravenously.

(1). 1×10⁵ tumor cells or primary normal cells were seeded in a 24-well culture plate with 10% FBS DMEM medium. The cells numbered 2×10⁵ next day. Almost 70% of the cells were confluent. The liquid was discarded. 100 μl of adenovirus filtrate (about 10³ adenoviruses) was added to each well and was shaken gently 3 times. The cells mixture was incubated in 5% CO₂ at 37° C. for 90 minutes.

To the mixture was added a culture medium containing 1% FBS DMEM to have the volume reach 1 ml. The cell mixture was incubated in 5% CO₂ at 37° C. for 3-10 days. The time when the complete CPE of the cells appeared was observed.

The result was shown in Table 1 from which the wild-type adenovirus ADV5 as the positive control unselectively lysed all the tumor and normal cells, the replication-deficient adenovirus (ADV-TK) didn't lyse any cells tested. The recombinant adenovirus constructs Δ923-931ADV5/ASCHK1 and Δ923-931ADV5/ASPLK1 showed selective lysis of tumor cells, the effect of which was higher than that of the wild-type ADV5, but no prominent effects on the normal control cells.

TABLE 1

| | | | Δ923-931 | Δ923-931 |
|---|---|---|---|---|
| | | | ADV5/ | ADV5/ |
| | Wild-type ADV5 | ADV-TK | ASCHK1 | ASPLK1 |
| A549 | | | | |
| 3rd day | 50% | 1% | 93% | 95% |
| 6th day | 100% | 2% | 100% | 100% |
| 10th day | | 4% | | |
| Hela | | | | |
| 3rd day | 60% | 1% | 94% | 90% |
| 6th day | 100% | 2% | 100% | 100% |
| 10th day | | 4% | | |
| U-2OS | | | | |
| 3rd day | 60% | 1% | 95% | 90% |
| 6th day | 100% | 2% | 100% | 100% |
| 10th day | | 4% | | |
| HS700T | | | | |
| 3rd day | 60% | 1% | 93% | 90% |
| 6th day | 100% | 2% | 100% | 100% |
| 10th day | | 4% | | |
| DLD-1 | | | | |
| 3rd day | 60% | 1% | 90% | 90% |
| 6th day | 100% | 2% | 100% | 100% |
| 10th day | | 4% | | |
| MCF-7 | | | | |
| 3rd day | 60% | 1% | 95% | 92% |
| 6th day | 100% | 1% | 100% | 100% |
| 10th day | | 1% | | |
| Vascular | | | | |
| endothelial cells | | | | |
| 3rd day | 60% | 1% | 4% | 4% |
| 6th day | 100% | 2% | 4% | 4% |
| 10th day | | 4% | 4% | 4% |
| Lung tracheole | | | | |
| endothelial cells | | | | |
| 3rd day | 60% | 1% | 3% | 3% |
| 6th day | 100% | 2% | 3% | 3% |
| 10th day | | 4% | 6% | 6% |
| prostate | | | | |
| endothelial cells | | | | |
| 3rd day | 60% | 1% | 4% | 4% |
| 6th day | 100% | 2% | 4% | 4% |
| 10th day | | 4% | 5% | 5% |
| Bone Marrow | | | | |
| Mononuclear | | | | |
| Cells | | | | |
| 3rd day | 60% | 1% | 2% | 2% |
| 6th day | 100% | 2% | 2% | 2% |
| 10th day | | 4% | 6% | 6% |

(2). Experiment was performed as follows to further quantitatively determine the replication efficiency of the recombinant adenovirus construct.

1×10⁵ tumor cells or primary normal cells were seeded in a 24-well culture plate in 10% FBS DMEM medium. The cells were numbered 2×10⁵ next day. Almost 70% of the cells were confluent. The liquid was discarded. 100 μl of the recombinant adenovirus (10 MOI diluted) was added to each well and shaken gently 3 times. The cells were incubated in 5% CO₂ at 37° C. for 90 minutes.

1% FBS DMEM was added till the volume of 1 ml. The cells were incubated in 5% CO₂ at 37° C. for 6 days.

300 μl of PBS solution was added. The mixture was frozen/thawed for 3 cycles to release the adenovirus. The lysed solution was centrifuged for 10 minutes. The supernatant was collected and kept at −80° C. The titer of the adenovirus was determined by TCID₅₀ in 293 cells.

The results showed that the potency of Δ923-931ADV5/ASCHK1 in tumor cells after replication was 400-1000 folds as the original, and that of Δ923-931ADV5/ASPLK1 was 100-1000 folds as the original, while the infection potency of both in normal cells represented no evident changes. The results showed that the recombinant adenovirus construct specifically replicated in tumor cells.

2. Expression of In Vitro Selectively Inactivating Tumor Genes of Recombinant Adenovirus Construct

(1). The objective of the experiment is to identify the effects of recombinant adenovirus Δ923-931ADV5/ASCHK1 upon the CHK1 expression in various tumor and normal cells. Cell lines used in this experiment were selected from A549, Hela, human osteoblastoma U-2OS cell line (p53+, RB−, human metastatic adenoma HS700T cell line (p53−, RB−) and human colon cancer MCF-7 cell line (p53+, RB+).

(2). 1×10⁵ tumor cells or primary normal cells were seeded in a 24-well culture plate with 10% FBS DMEM medium. The cells numbered 2×10⁵ next day. Almost 70% of the cells were confluent. The liquid was discarded. 100 μl of adenovirus filtrate (about 10³ adenoviruses) was added to each well and was shaken gently 3 times. The cells mixture was incubated in 5% CO₂ at 37° C. for 90 minutes.

To the mixture was added a culture medium containing 1% FBS DMEM to have the volume reach 1 ml. The cell mixture was incubated in 5% CO₂ at 37° C. for 3-10 days. The total cell protein was extracted to determine a change of CHK1 protein expression level.

The result was showed in Table 1 from which the wild-type adenovirus ADV5 as the positive control unselectively lysed all the tumor and normal cells, the replication-deficient adenovirus (ADV-TK) didn't lyse any cells tested. The recombinant adenovirus constructs Δ923-931ADV5/ASCHK1 and Δ923-931ADV5/ASPLK1 showed selective lysis of tumor cells, the effect of which was higher than that of the wild-type ADV5, but no prominent effects on the normal control cells.

The results showed that the recombinant adenovirus construct Δ923-931ADV5/ASCHK1 selectively inactivated CHK1 protein expression of tumor cells up to an undetectable extent, and the recombinant adenovirus construct Δ923-931ADV5/ASPLK1 selectively inactivated PLK1 protein expression of tumor cells to an undetectable extent. But both of them had no prominent effects on the normal control cells. The result demonstrated that the recombinant construct of the invention selectively inactivated tumor gene expression.

3. Anti-Tumor Activity of Recombinant Adenovirus Construct In Vivo

Tumor Cell Animal Model

A female athymic nu/nu mouse with an age of 5 weeks having good growth of MCF-7 tumor and good health status was killed with cervical dislocation. Tumor clumps were scalped under a sterilized condition and were sliced in a 1-2 mm diameter. The tumors were transplanted by trocar needles into both oxters of nude mice. After about one week, the tumor appeared in both oxters.

(1). Administration via Direct Injection

When the tumor grew with a 6-7 mm diameter, 1×10⁸ pfu adenovirus was directly injected into the tumor, and it lasted for 5 days. The length and width of the tumor treated with Δ923-931ADV5/ASCHK1 and Δ923-931ADV5/ASPLK1 were then measured with a vernier caliper for 90 days or till the tumor volume more than 1 cm³.

The results showed that at the end of the experiment (90 days), the inhibition rate against the MCF-7 tumor of the wild-type ADV5 as a positive control was 80%±2%, that of ADV-TK as a negative control was −50%±2%, and that of the recombinant adenovirus constructs Δ923-931ADV5/ASCHK1 92%±4% and Δ923-931ADV5/ASPLK1 was 95%±4%. The result showed that the recombination adenovirus construct of the invention had significant anti-tumor activity in vivo.

(2). Administration via Vein

When the tumor grew with a 4-5 mm diameter, 1×10⁸ pfu adenovirus was intravenously injected into the tumor, and it lasted for 5 days. The length and width of the tumor treated with Δ923-931ADV5/ASCHK1 and Δ923 -931ADV5/ASPLK1 were then measured with a vernier caliper for 60 days or till the tumor volume more than 1 cm³.

The results showed that at the end of the experiment (60 days), the inhibition rate against the MCF-7 tumor of the wild-type ADV5 as a positive control was 60%±2%, that of ADV-TK as a negative control was −70%±2%, and that of the recombinant adenovirus constructs Δ923-931ADV5/ASCHK1 was 95%±4% and Δ923-931ADV5/ASPLK1 was 96%±4% The result showed that the recombination adenovirus construct of the invention had significant anti-tumor activity by intravenous administration.

4. Inhibition of Tumor Metastasis of Recombinant Adenovirus Construct

Tumor Cell Animal Model:

A female athymic nu/nu mouse with an age of 5 weeks of having good growth of MDA-MB-321 (ATCC, USA, Catalogue No. HTB-26) and good health status was killed with cervical dislocation. Tumor clumps were scalped under a sterilized condition and were sliced in a 1-2 mm diameter. The tumors were transplanted by trocar needles into the breast of nude mice. After about one week, the tumor appeared in both oxters.

Administration via Intravenous Injection:

When the tumor grew with a 4-5 mm diameter, 1×10⁸ pfu adenovirus was intravenously injected into the tumor, and it lasted for 5 days. The length and width of the tumor treated with Δ923-931ADV5/ASCHK1 and Δ923-931ADV5/ASPLK1 of the invention were then measured with a vernier caliper for 90 days or till the tumor volume more than 1.2 cm³. Meanwhile, trachea and level scrofula of the tested animal were taken for assaying tumor metastasis.

The results showed that at the end of the experiment (90 days), the inhibition rate against the tumor of the wild-type ADV5 as a positive control was 60%±2% and the rate of tumor metastasis was 45%±5%, the inhibition rate against the tumor of ADV-TK as a negative control was −70%±2% and the tumor metastasis rate was 100%, and the inhibition rate against the tumor of the recombinant adenovirus constructs Δ923-931ADV5/ASCHK1 and Δ923-931ADV5/ASPLK1 were 94%±4% and 93%±5%, respectively, and the tumor metastasis rate of treatment with either construct was 0. The results showed that the recombination adenovirus constructs of the invention had significant anti-tumor activity and inhibiting activity of tumor metastasis in vivo via intravenous administration.

1. A recombinant construct of an ADV5, comprising a first deletion in the E1A region of the ADV5 genome, and a second deletion in the E3 region, wherein a foreign cDNA fragment is inversely inserted into the second deletion.

2. The recombinant construct of claim 1, wherein the first deletion is from nucleotide No. 923 to nucleotide No. 931 of the ADV5 genome.

3. The recombinant construct of claim 1., wherein the second deletion is from nucleotide No. 28532 to nucleotide No. 29360 of the ADV5 genome.

4. The recombinant construct of claim 2, wherein the second deletion is from nucleotide No. 28532 to nucleotide No. 29360 of the ADV5 genome.

5. The recombinant construct of claim 1, wherein an enzyme cleavage site is introduced into the second deletion and the foreign cDNA fragment is inversely inserted into the enzyme cleavage site.

6. The recombinant construct of claim 5, wherein said cleavage site is ClaI.

7. The recombinant construct of claim 1., wherein the foreign cDNA fragment is a gene necessary for survival of a cell.

8. The recombinant construct of claim 4, wherein the inserted foreign cDNA is selected from a CHK1 cDNA fragment and a PLK1 cDNA fragment.

9. The recombinant construct of claim 8, wherein the CHK1 cDNA fragment corresponds to nucleotide No. 853 to nucleotide No. 250 of CHK1 mRNA.

10. The recombinant construct of claim 8, wherein the PLK1 cDNA fragment corresponds to nucleotide No. 960 to nucleotide No. 161 of PLK1 mRNA.

11. A method for preparing a recombinant ADV5 construct of claim 1, comprising:
deleting a fragment in the E1A region of an ADV5 genome;
deleting a fragment in the E3 region of the ADV5 genome; and
inversely inserting a foreign cDNA fragment into the deleted E3 region.

12. The method of claim 11 wherein the deleted fragment in the E1A region is from nucleotide No. 923 to nucleotide No. 931 of the ADV5 genome.

13. The method of claim 11 wherein the deleted fragment in the E3 region is from nucleotide No. 28532 to nucleotide No. 29360 of the ADV5 genome.

14. The method of claim 12 wherein the deleted fragment in the E3 region is from nucleotide No. 28532 to nucleotide No. 29360 of the ADV5 genome.

15. A method of claim 11, further comprising introducing an enzyme cleavage site into the deleted E3 region, and inversely inserting the foreign cDNA fragment into the enzyme cleavage site.

16. The method of claim 15, wherein the enzyme cleavage site is ClaI.

17. The method of claim 11, wherein the foreign cDNA fragment is selected from a CHK1 cDNA fragment and a PLK1 cDNA fragment.

18. The method of claim 12, wherein the CHK1 cDNA fragment corresponds to nucleotide No. 853 to nucleotide No. 250 of CHK1 mRNA.

19. The method of claim 12, wherein the PLK1 cDNA fragment is corresponds to nucleotide No. 960 to nucleotide No. 161 of PLK1 mRNA.

20. A method of a recombinant ADV5 construct, comprising the steps of:
a) deleting a 923-931 nt sequence from the E1A coding region of a plasmid pXC1 to form a first vector;
b) co-infecting a first cell with the first vector and pBHGE3;
c) extracting a DNA containing terminal proteins from the infected cell and digesting the DNA with EcoRI to obtain a first fragment;
d) deleting a 28532-29360 nt sequence from the E3 region of an ADV5 and inserting a enzyme cleavage site in the deleted E3 region to form a second vector;
e) inversely inserting a foreign cDNA fragment in the enzyme cleavage site to form a third vector;
f) digesting the third vector with EcoRI to obtain a second fragment; and
g) co-infecting a second cell with the first fragment and the second fragment to obtain the recombination adenovirus construct that expresses an E1A mutant incapable of binding to Rb, but capable of activating transcription.

21. The method of claim 20, wherein the cleavage site is ClaI.

22. The method of claim 20, wherein the foreign cDNA fragment is selected from a CHK1 cDNA fragment and a PLK1 cDNA fragment.

23. The method of claim 22, wherein the CHK1 cDNA fragment corresponds to nucleotide No. 853 to nucleotide No. 250 of CHK1 mRNA.

24. The method of claim 22, wherein the PLK1 cDNA fragment corresponds to nucleotide No. 960 to nucleotide No. 161 of PLK1 mRNA.

25. The method of claim 20, wherein both said first and second cells are 293 cells.

26. A method of treating a tumor in a subject comprising administrating to a subject having a tumor a therapeutically effective amount of a recombinant ADV5 construct of claim 1.

27. The method of claim 26 wherein the recombinant ADV5 construct is intravenously administrated to the subject.

28. The method of claim 26 wherein the recombinant ADV5 construct is directly injected to the tumor of the subject.

29. A method for inhibiting tumor metastasis in a subject comprising administrating to a subject having a tumor a therapeutically effective amount of a recombinant ADV5 construct of claim 1.

30. The method of claim 28 wherein the recombinant ADV5 construct is intravenously administrated to the subject.

31. The method of claim 30 wherein the recombinant ADV5 construct is directly injected to the tumor of the subject.
